(19) United States
(12) Patent Application Publication
(10) Pub. No.: US 2005/0124848 A1
(43) Pub. Date: Jun. 9, 2005

(54) Method and apparatus for electromagnetic modification of brain activity

(76) Inventor: Oliver Holzner, Bruck (DE)

Correspondence Address: HOUSTON ELISEEVA, 4 MILITIA DRIVE, SUITE 4, LEXINGTON, MA, 02421, US

(21) Appl. No.: 10/959,033

(22) Filed: Oct. 5, 2004

Related U.S. Application Data
(63) Continuation of application No. PCT/EP03/03545, filed on Apr. 4, 2003.

(30) Foreign Application Priority Data
Apr. 5, 2002 (DE) DE 102 15 115.6
Jul. 30, 2002 (DE) DE 102 34 676.3

(51) Int. Cl. A61B 17/52; A61N 2/00

(52) U.S. Cl. 600/9; 600/544; 607/1

(57) ABSTRACT

A method and an apparatus for the electromagnetic modification of brain activity, in particular for the model-based controlled or regulated, respectively, electromagnetic modification of brain activity in vivo, as well as to the resulting modification of behavior is disclosed. A brain activity model (which describes the influence of exogenous electric and/or magnetic fields on a brain activity) is used, and a behavioral model (which describes the correspondence between brain activity and behavior) is used. Thereby it becomes possible, for the first time ever, to influence the behavior of a person by means of exogenous input in a controlled way. Intra-individual and/or time-dependent non-observables, as well as to determining individual, is provided, if necessary intra-individual and/or time-dependent translation operators from extracranial signal to control force, whereby a secure and controlled intervention as part of a open or closed control loop is created, which in turn results in achieving a brain activity target in a reliable way. Using a behavioral model ensures that achieving a brain activity target corresponds to achieving the individual behavioral target of the user.

### RELATED APPLICATIONS

This application is a Continuation of PCT application serial number PCT/EP03/03545 filed on Apr. 4, 2003 (which was published in German under PCT Article 21(2) as International Publication No. WO 03/085546 Al), which claims priority to German Application Nos. DE 102 34 676.3 filed on Jul. 30, 2002 and DE 102 15 115.6 filed on Apr. 5, 2002, all three applications being incorporated herein by reference in their entirety.

This application is related to U.S. Application No.: (Attorney Docket No. 0001.0013US1 (US-5440)) filed on even date herewith by Oliver Holzner, entitled “Method and Apparatus for the Prevention of Epileptic Seizures,” which is also incorporated herein by reference in its entirety.

### BACKGROUND

Definitions:

MODEL-BASED controlled or regulated, respectively, modification means alteration or sustenance of brain activity, based on: a behavioral target, a behavioral model, and a brain activity model.

Regulated means, that measurement-, calculation-, and modification steps are interconnected in feedback loops.

The BEHAVIORAL TARGET set by the user includes type, intensity, and duration of a particular behavior.

BEHAVIOR relates to any spatio-temporal combination of any existing or possible states of a person, which can potentially be validated. Behavior includes, but is not limited to, perception, actions, motivation, focused attention, memory, learning, consciousness, emotions, cognition, mental states, abilities, personality traits.

Behavior is based on brain activity. The correspondence between both is quantified within the framework of a behavioral model, i.e. an empirically validated relation between certain behavior and certain dynamics of certain brain activity indexes.

BRAIN ACTIVITY INDEXES are characteristics for brain activity calculated from measured data. Examples for brain activity indexes are voltage differences between an EEG-electrode and a reference electrode, or a frequency band of the most prominent frequencies in these data for all EEG electrodes (“alpha-band” or similar indexes [12]), or the similarity index (see [5]) of the values measured by MEG-sensors.

A BRAIN ACTIVITY MODEL relates to a physiologically based model, which describes brain activity via the dynamics of brain constituents and/or their interactions (this includes, e.g. [24], [1], [23], [25], or [18]).

Conventional models are not specific, but GENERIC, because in them non-observables are estimated via mean values of populations.

A NON-OBSERVABLE is a parameter or a variable of a generic model, which can, in-vivo, either only be measured with substantial efforts, or cannot be measured at all (with the necessary spatial and temporal resolution). Non-observables have a decisive influence on brain dynamics. They fluctuate between different individuals and within one individual up to a factor of 100. Generic brain activity models make no statements about direct exogenous influence on the dynamics of brain constituents. In the best case they make statements about indirect exogenous influence via receptor stimulation, deprivation, or lesions.

Under normal circumstances, in the following specific brain activity models will be used, which include exogenous inputs via electrical and/or magnetic fields. These models are based on non-observables relevant for the specific user within a time-interval, which have been determined with suitable methods. These brain activity models normally include for each brain constituent non-linear differential equations of the type
x(t)=ƒ(x,t,localParameters, endogenousInput, exogenousInput)

Here x is a brain activity index, the dot indicates its time derivative, t is time, f a function of, amongst other things, the type of constituent (“local parameters”), the type and scope of the influence of other constituents projecting into the constituent under consideration (“endogenous input”), and the direct exogenous input on the respective constituent (“exogenous input”). Endogenous input includes, depending on the model used, further (“translocal”) parameters, like, e.g. coupling strengths or transmission delays between constituents.

Missing or incomplete specification of the brain activity model with respect to the particular user leads to a wide variation of, in this case, mostly unknown consequences of exogenous electromagnetic input, and therefore to non-applicability of related methods outside the realm of neurological, psychiatric, or psychological defects, in which random consequences are to be weighed against the possibility of successful treatment.

ELECTROMAGNETIC means electric and/or magnetic observables and relates to the type of measured data (essentially electromagnetic correlates of the user's brain activity), as well as to the main mode of modification (e.g. via a variable magnetic field, generated extracranially, which penetrates the cranium of the user without problem, and intracranially generates induced potentials, whereby it becomes possible to influence the brain activity of the user.)

IN VIVO relates to the application of the method to living users, in contrast to cell cultures, brain slices, computer-simulations, and the like. This therefore is particularly demanding with respect to the complexity of actual brain processes occurring, as well as to measurement-, calculation-, and modification speed, as well as to the safety of the method.

Related Art:

At the current state of the art model-based controlled or regulated, respectively, electromagnetic modification of brain activity in vivo is not possible (the human brain consists of approximately 1011 neurons and 1015 connections between these neurons), and has so far neither been considered nor attempted. The same applies to related behavioral modification.

There are three hardly overlapping scientific/technical directions of approaches related to brain activity respectively to altering brain activity:
a nonlinear dynamic/neural networks/artificial intelligence approach (in the following denoted as “NA”), in which certain features of the living human brain shall be mimicked in simplified replications, in order to build machines with similar features (so-called “neurocomputers”),
a control-theoretic approach (in the following denoted as “CON”), in which principles of mathematical control theory are applied to very specific parts of a brain-computer-entity,
a medical approach (in the following denoted as “MED”), concerned with practical/clinical applications of electromagnetic fields of (mainly) high field strengths on humans or animals.

A possible constituent of NA is the NEURAL OSCILLATOR, i.e. an entity which may switch between oscillating and non-oscillating behavior, and which may comprise smaller entities (which are not considered any further). NA is concerned with neural networks, i.e. small (of the order of <10⁴) connected ensembles of neural oscillators, and the phenomena, which occur in these networks (e.g. memory or pattern recognition). After originally basic principles of functioning were extracted from nervous systems of living organisms, NA now proceeds towards implementation of these principles in artificial systems. Exemplary for this development is [2].

After starting out with applying linear control to sufficiently simple mechanical systems, CON proceeds into the opposite direction. To apply linear control to a nonlinear respectively stochastic system (like e.g. a human brain), is not only inadequate, but also potentially dangerous, because in linear control the control force is proportional to the desired change. It may therefore become very large.

Control methods can be categorized into model-based and data-based methods. The quality of a model-based method is related to the suitability of the model with respect to the problem under consideration. So far neither for humans nor for animals brain activity models or behavioral models have been applied.

The quality of a data-based method depends on the simplicity of the system-to-be-controlled (because the first step in a data-based method tends to be the reconstruction of the phase space, in which, later on, searches are conducted, and iterations are calculated—which is technically impossible to carry out in case of higher-dimensional phase spaces. Exception is time-delayed feedback, which, however, involves waiting times until the target-state to be stabilized is reached). For a controlled or regulated, respectively, electromagnetic modification of brain activity in vivo current control methods are not applicable, because several of the following effects accumulate:
between high and unachievable high demands on storage space and processor speed,
potentially infinitely long waiting times until the target orbit is reached,
in systems with stochastic elements intermittent departures from the target orbit,
restrictions on the range of control in systems, in which only aggregated behavior is measurable,
restrictions on the range of control in systems, in which the control force is smeared,
unknown amplitude of the control force within heterogeneous systems.

MED is implicitly based on a microscopic physiological model with single neurons as constituents of brain activity, which shall in the following be called SINGLE NEURONS. Consequently, in vivo non-invasively non-observable variables like ion concentrations on both sides of the cell membranes, number of dendrites etc. play an essential role for calculating the number of neuron firings per time unit (firing rate). For an automatic, individualized, controlled or regulated, respectively, electromagnetic modification of brain activity in vivo the single-neuron-paradigm (with its 10¹¹ neurons and 10¹⁵ connections between these neurons) is unsuitable. Therefore the MED approach based on this paradigm is hampered by a seemingly inexplicable randomness of its clinical results (for transcranial magnetic stimulation see [3] as an overview). Further MED methods, which have an impact on brain activity, are intra- and extracranial electric shocks. All MED methods are targeted at depolarizing neural membranes, and thereby at generating more action potentials (so-called “stimulation”).

In CON-terminology the electromagnetic depolarization of single neurons by MED-methods can be described as “slaving” (sensory slaving by rhythmic light flashes, acoustic signals, or the like, shall not be considered). Slaving is the result of applying artificial signals of high intensity to a system, which, during this application, will adopt the exogenous signal pattern instead of its natural behavior. In NA it is known, that under weak coupling in neural networks communication depends on commensurateness of the frequencies, with which the neural constituents oscillate. Therefore local slaving with an incommensurate or weakly commensurate frequency, will remove such slaved neurons from their normal communication, and thereby generate a virtual lesion. Current MED-methods consider virtual lesions as the main effect of TMS—an effect which is inexplicable in the single-neural paradigm.

MED-methods consider a “stimulation” as improved, if a less high intensity can be used to make the single neurons fire, or if the slaving area can be circumscribed more narrowly. Repeated slaving at the same medically relevant locus may lead to physiological changes, the duration of which is longer than the duration of the application, which, in some cases, is positive (e.g. statistically significant improvement of certain types of clinical depression under repetitive TMS, see e.g. [3]).

When applied to humans, there are only two partial aspects of current methods which are individualized: firstly, finding a suitable locus of the single neurons to be depolarized, secondly, finding a reference value for the strength of the slaving electromagnetic field (quoted in percent of a “motor threshold”). In a more recent MED method [4], TMS is used for diagnostic purposes, in order to gain information about the existence or non-existence of physiological connections between different areas of the brain within a particular individual, as well as (under certain restrictions) transmission speeds between different brain areas.

Current MED methods are preferably carried out for medical purposes, for which the disadvantages of slaving (including the remote possibility of seizures) are at least partially compensated for by their medical effects. Current MED methods include only passive safety measures, like avoiding high frequency slaving, exclusion of users prone to seizures, switching off the field, if a seizure should occur.

### SUMMARY OF THE INVENTION

In summary: With current techniques and methods, a model-based, controlled or regulated, respectively, electromagnetic modification of brain activity in vivo can neither be achieved, nor are these techniques and methods proceeding towards achievability: MED is stuck in the single neuron paradigm, NA proceeds towards building artificial systems, and CON is suffering from in-vivo-inapplicability to complex heterogeneous systems.

The invention relates to a method and an apparatus for the electromagnetic modification of brain activity, in particular for the model-based controlled or regulated, respectively, electromagnetic modification of brain activity in vivo, as well as to the resulting modification of behavior.

Object of the present invention is to create a method and an apparatus for the controlled or regulated, respectively, electromagnetic modification of brain activity in vivo, as well as the resulting behavioral modification.

In the method according to the present invention for the electromagnetic modification of brain activity a brain activity model (which describes the influence of exogenous electric and/or magnetic fields on a brain activity) is used, and a behavioral model (which describes the correspondence between brain activity and behavior) is used. Thereby it becomes possible, for the first time ever, to influence the behavior of a person by means of exogenous input in a controlled way.

In particular, the invention relates to using a brain activity model, as well as to determining individual, if necessary intra-individual and/or time-dependent non-observables, as well as to determining individual, if necessary intra-individual and/or time-dependent translation operators from extracranial signal to control force, whereby a secure and controlled intervention as part of a open or closed control loop is created, which in turn results in achieving a brain activity target in a reliable way. Using a behavioral model ensures that achieving a brain activity target corresponds to achieving the individual behavioral target of the user.

The invention is based on the knowledge, that behavioral models quantify the correspondence of behavior and the dynamics of brain activity indexes, and that suitable individualized brain activity models quantify the dynamics of brain activity indexes and state suitable control parameters, such that a reliable intervention in order to achieve an individual behavioral target is feasible.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

The invention will, in the following, be explained by way of examples, using the figures. The figures show:

FIG. 1 a transmitter seen in a sectional view,

FIG. 2 the transmitter of FIG. 1 seen from below,

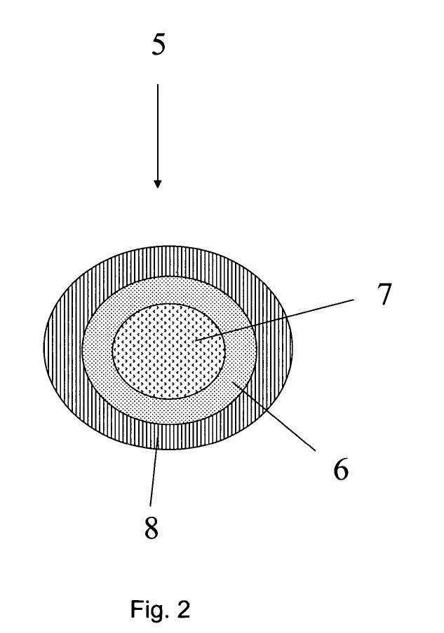

FIG. 3 a planar projection of drill-holes for sensors and transmitters reflecting their locations within a helmet,

FIG. 4 a helmet with overhead suspension and chin-rest,

FIG. 5 another planar projection of drill-holes for sensors and transmitters according to their locations within a helmet,

FIG. 6 the schematic interaction of head unit, intermediate unit, and basis unit,

FIG. 7 BAI-model-modes under variation of τ (extracts),

FIG. 8 BAI-model-modes under variation of a (extracts),

FIG. 9 BAI-model-modes under variation of b (extracts),

FIG. 10 BAI-model-modes under variation of c (extracts),

FIG. 11 BAI-model-modes under variation of d (extracts),

FIG. 12 Similar BAI-model-modes for two different model-modes I and II (example),

FIG. 13 BAI-model-modes for I under sine-shaped exogenous inputs of different strengths (examples),

FIG. 14 BAI-model-modes for II under sine-shaped exogenous inputs of different strengths (examples),

FIG. 15 BAI-model-modes for I under exogenous elementary loops of low strengths (examples),

FIG. 16 BAI-model-modes for II under exogenous elementary loops of low strengths (examples),

FIG. 17 BAI-model-modes for I under exogenous elementary loops of medium strengths (examples),

FIG. 18 BAI-model-modes for II under exogenous elementary loops of medium strengths (examples),

FIG. 19 a theta-BAI-model-mode under different endogenous inputs and one exogenous input (examples),

FIG. 20 a flow diagram of the inventive method,

FIG. 21 a flow diagram of the method S2000 (calibration),

FIG. 22 a flow diagram of the method S2100 (local calibration, 1st part),

FIG. 23 a flow diagram of the method S2100 (local calibration, 2nd part),

FIG. 24 a flow diagram of the method S2300 (translocal calibration),

FIG. 25 a flow diagram of the method S3000 (control and/or regulation, respectively, of brain activity),

FIG. 26 a diagram, which shows data from one EEG-channel,

FIG. 27 a diagram, which shows a phase-space representation of parts of the data from FIG. 26,

FIG. 28 a diagram, which shows a phase-space representation of neural activity under various inputs, modeled with the Wilson-Cowan model,

FIG. 29 a diagram, which shows a phase-space representation of neural activity with the same input, under different mixing angles, modeled with the Wilson-Cowan model.

### DETAILED DESCRIPTION OF THE PREFERRED EMBODIMENTS

Apparatus:

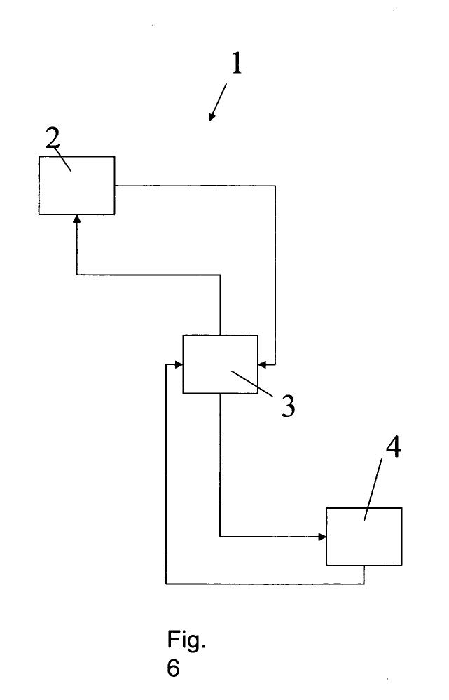

An apparatus 1, which as been constructed according to the principles of the present invention, comprises
one or more head-units 2, connected with
an intermediate unit 3, of which one or more are connected with
a basis unit 4 (FIG. 6).

A head-unit 2 includes a measurement system with devices (“sensors”) for measuring electromagnetic data, devices for data-preprocessing, and data transfer to an intermediate unit, as well as a regulation system with devices (“transmitters”) 5 for generating electromagnetic fields, as well as for carrying out digital control- or regulation-instructions (arriving from the intermediate unit) by means of transmitter signals. An intermediate unit includes a computer with software, by which the methods for controlling brain activity described below (in case calibration is not necessary) are embodied, as well as connections to and from the basis unit. The basis unit 4 includes a computer, which firstly contains a data base with model- and user-data, and on which secondly further methods described below in more detail are implemented. Intermediate- and basis-unit are localized on the same or on different computers.

An embodiment of the method comprises one head-unit, one intermediate unit, and one basis-unit. The head unit includes e.g. a measurement system with an EEG-cap with its extracranial sensors, connections to amplifier, amplifier, connections to A/D-converter, A/D-converter, connections to intermediate unit, and a control system with extracranial induction coils as transmitters, controllable power sources for these transmitters, connections, D/A-converter, connection with the intermediate unit. The intermediate unit includes e.g. a personal computer with screen and keyboard, as well as software, as well as connections to and from the basis unit. The basis unit includes e.g. a powerful computation unit.

Suitable sensors are e.g. EEG-or MEG-sensors. The MEG-sensors comprise e.g. a SQUID-sensor-element with suitable processing devices for the detection of a magnetic field, as well as cooling devices. The EEG-sensors comprise e.g. two electrodes for measuring a difference in electrical potential.

A preferred embodiment of a sensor includes a partial or complete electric and/or magnetic shielding of this sensor with respect to its surroundings, without obstructing its functionality.

A preferred embodiment of the measurement system within a head unit may contain a plurality of sensors, located intra- or extracranially. This plurality of sensors is called sensor grid.

A preferred embodiment of an extracranial sensor grid includes fittings such that, after taking the sensor grid on and off several times, the sensors will resume their previous relative positions with respect to the cranium of the respective user. This is achieved, e.g., by fitting the sensor grid to the inside of a helmet, the inside of which is fitted to the cranial shape of the user.

Another preferred embodiment of a sensor grid includes implanted electrodes.

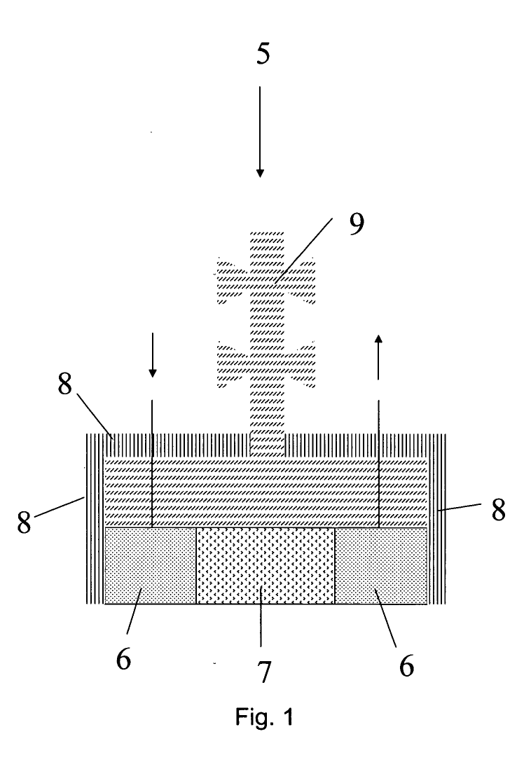

A preferred embodiment of an extracranial transmitter 5 includes a conducting coil 6 with para-, dia-, or ferro-magnetic core 7, as shown in FIG. 1 from a sectional view, with each arrow symbolizing the direction of the flow of electric current. The transmitter 5 has essentially a cylindrical shape, with face- and side-area covered by a shielding 8. Coil 6 and core 7 are immediately adjacent to that side of transmitter 5, which has no shielding. When operating the device, this side points towards the cranium, in order to transmit exogenous magnetic fields. On the backside of transmitter 5 a fitting 9 can be found, whereby the transmitter 5 can be fitted into a helmet.

The extracranial transmitter 5 may include protection against deformation, e.g. by embedding the conducting parts into suitable resin, or embedding the conducting parts into stable insulating material.

The extracranial transmitter 5 may include cooling devices.

Another preferred embodiment of an intracranial transmitter includes implanted electrodes.

A preferred embodiment of the invention relating to the control parts of the head unit includes a plurality of transmitters, distributed intra- and/or extracranially. This plurality is called transmitter grid.

A preferred embodiment of an extracranial transmitter grid includes fittings with respect to the cranium of the user, such that, after taking the transmitter grid on and off several times, the transmitters will resume their previous relative position. This is achieved, e.g., by fitting the transmitter grid to the inside of a helmet, which is fitted to the cranial shape of the user.

In an alternative embodiment the locking of the position of sensor grid and/or transmitter grid with respect to the cranium of the user is camera-assisted, whereby the spatial position of the user's head, as well as the relative positions of sensor- and transmitter-grid are captured by several cameras, the data of which are processed to 3D-data.

In another embodiment, sensors and/or transmitters are implanted electrodes, with which EEG measurements may be performed, and with which currents may be conducted into the brain. Leads connecting these electrodes and/or their interfaces with the intermediate unit and/or further leads and/or further measurement devices and/or the related computer and/or power sources for the electrodes and/or the computer may also be implanted, whereby a fully mobile mode of operation is feasible.

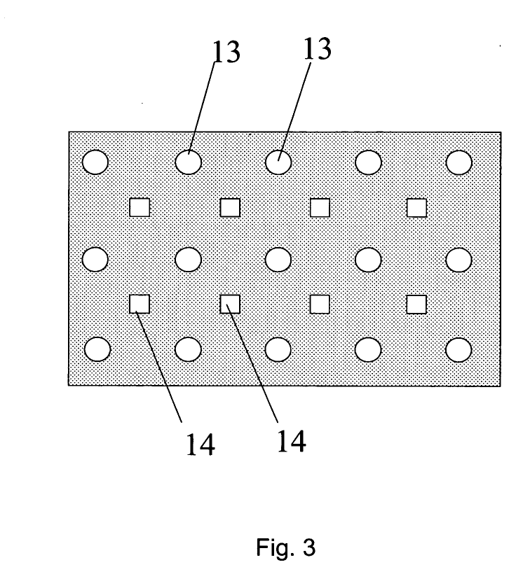

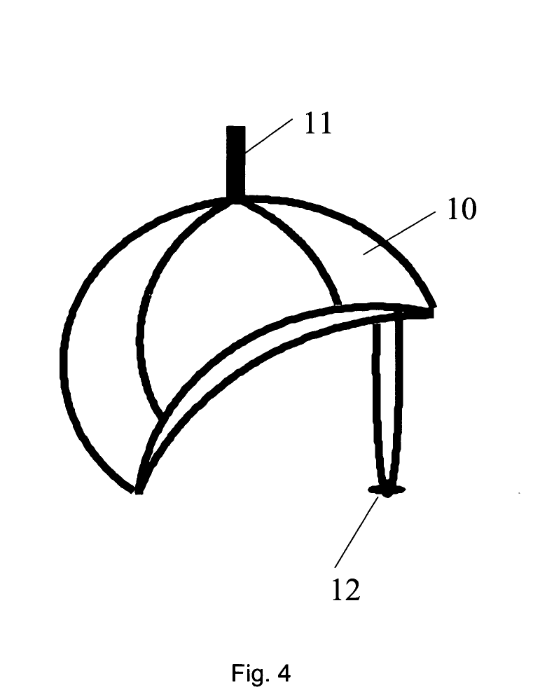

In FIG. 4 a preferred embodiment of the invention relating to the head unit of an extracranial measurement- and control-system is shown schematically, which comprises a helmet 10, which fits the cranial shape of the respective user, together with a cylindrical overhead suspension 11 with connecting leads inside of it, and a chin-rest 12. Sensor- and transmitter-grid on the inside of the helmet are fitted in such a way, that both grids are superposed, i.e. in the vicinity of each sensor sufficiently many transmitters are located, and vice versa. A planar projection of a mechanical fitting for this embodiment is shown in FIG. 3 (drill-holes 13 for sensors are shown as circles, drill-holes 14 for transmitters 5 as squares). In the embodiment described the user sits on an armchair with a neck-rest below the helmet 10.

In an alternative embodiment of the invention, the sensor grid is intracranial, and the helmet contains the extracranial transmitter grid.

In an alternative embodiment of the invention, the transmitter grid is intracranial, and the helmet contains the extracranial sensor grid.

In still another embodiment of the invention, both sensor- and transmitter-grid are intracranial.

In a preferred embodiment of the invention sensor-density, as well as sensor-configuration of an extracranial sensor-grid are adjustable. In another preferred embodiment of the invention the adjustment is carried out automatically, controlled by the intermediate unit.

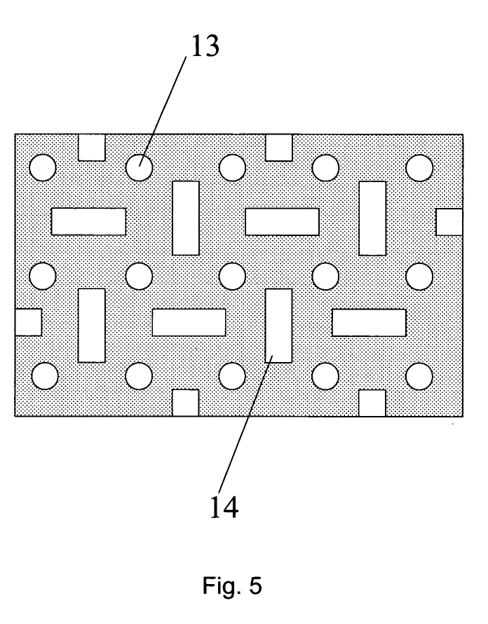

In a preferred embodiment of the invention transmitter-density, as well as transmitter-configuration of an extracranial transmitter-grid and/or the orientation of transmitters with respect to the cranium of the user are adjustable. A planar projection of a mechanical fitting of this embodiment of the invention is shown in FIG. 5. Here drill-holes 13 for sensors are shown as circles, drill-holes 14 for transmitters as squares. Here it is possible to fit transmitters into the drill-holes 14 of the fittings, and/or tilt transmitters 5 with respect to the fittings. Amongst other coil configurations, all conventional coil configurations with their spatial distributions, orientations, and field directions may be emulated.

Preparation, testing, and maintenance of the extracranial device are carried out by technicians, who are not required to have any medical education.

In a preferred embodiment of the invention, the apparatus contains conventional protection against power failure and/or voltage fluctuations.

At the intermediate unit real-time automatic procedures may be performed, both for density- and position-optimization of sensors and transmitters, and for artifact-removal, i.e. removal of measurement biases, which may be produced by exogenous magnetic fields, eye movements, muscle twitches, and others.

Method:

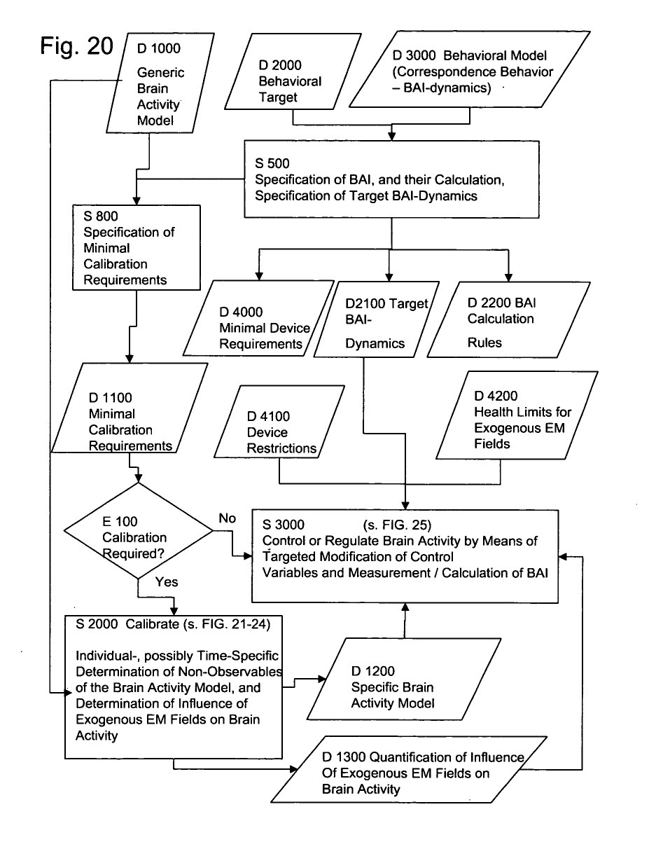

The method according to the present invention includes procedural steps, denoted by “S”, and explained below in detail. Input- and output-data are denoted by “D”. Each step will be explained in the following: The fundamentals of the method according to the present invention are shown in a flow diagram, FIG. 20, whereby rectangles symbolize procedural steps, rhombi decision steps, and parallelograms input- or output-data of procedural steps.

S 500:

In this step the variables necessary for achieving the behavioral target (D 2000) in a reliable way, as well as their dynamics are specified with the help of a behavioral model (D 3000).

The behavioral model models the correspondence between behavior and the dynamics of certain BAI. In the following, “BAI” denotes singular as well as plural of “brain activity index”. BAI are calculated from measured data according to specific rules (D 2200).

Examples for BAI are voltage differences, measured by a sensor (electrode plus reference electrode), or a power spectrum of a windowed signal originating from the data measured by this sensor, or the quotient of beta-divided-by-alpha EEG-activity for this sensor (beta=13-30 Hz, alpha=8-12 Hz), see [11].

An example for a behavioral model is the reduced Davidson-model (see e.g. [11]), whereby positive emotions correspond to a higher quotient of beta-divided-by-alpha EEG-activity in the left-frontal cortex (beta=13-30 Hz, alpha=8-12 Hz).

The behavioral model (D 3000) allows for transforming the behavioral target (D 2000), which in principle includes the duration of the desired behavior, into target dynamics of BAI (D 2100), and thereby for its quantification.

Type and number of BAI result in minimal device requirements (D 4000). For calculating e.g. the quotient of beta-divided-by-alpha EEG-activity in the left-frontal cortex (beta=13-30 Hz, alpha=8-12 Hz), at least one EEG-electrode plus one reference electrode is required. For achieving the target BAI in a controlled way at least one transmitter is required.

S 800

The BAI used are to be calculated from the variables of the generic brain activity model used (D 1000).

Example: BAI based on Fourier-decomposition for a circumscribed brain area can be calculated from the dynamics of neural oscillators of the Wilson-Cowan-model (see e.g. [1]).

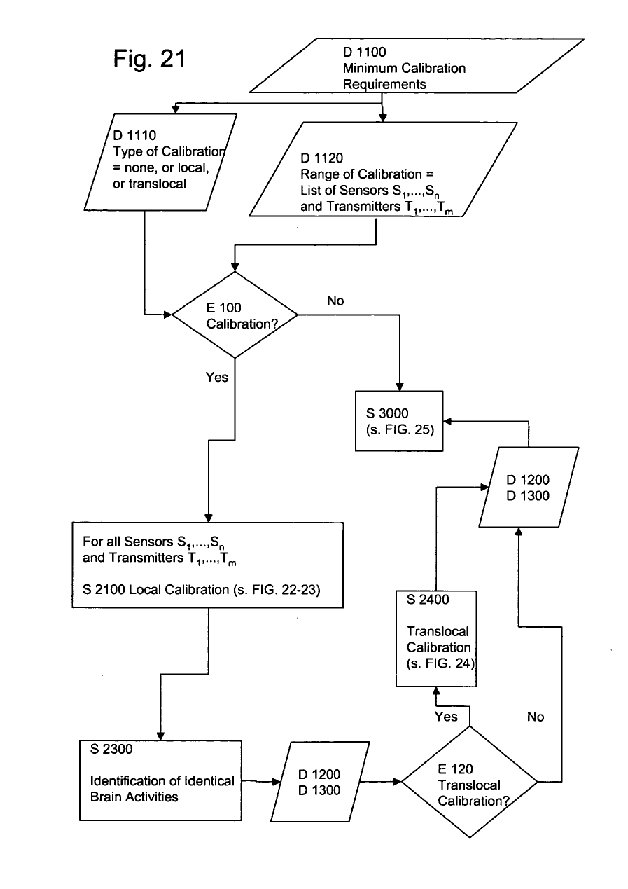

The BAI used result in minimal calibration requirements (D 1010) with respect to the generic model used. The less robust the calculation of BAI is, the higher are the requirements for calibration. If the BAI used are based on coupled non-robust variables in different locations, local calibration is to be augmented by non-local calibration. In any case, the type of calibration (e.g. “none”, “local”, translocal”, D 1110 in FIG. 21), and its range (e.g. list of sensors and transmitters) are to be listed (E 100).

Examples: for the robust, nonlinear similarity index (see e.g. [5]) a calibration is not absolutely necessary. For the abovementioned beta/alpha-quotient a local calibration of the Wilson-Cowan-model is required. For complex cognitive performance (e.g. musical creativity, see [19]), in general a translocal calibration will be necessary.

S 2000

Calibration includes the determination of individual, possibly time-dependent values of parameters (which are estimated by population mean values in the generic model, for endogenous input) and of the individual, possibly time-dependent influence of exogenous electromagnetic fields on brain activity (“electromagnetic” is used for “electric and/or magnetic”, and will, in the following, be abbreviated by “EM”). Range and quality of the calibration influence the reliability of modification of one or more brain activities.

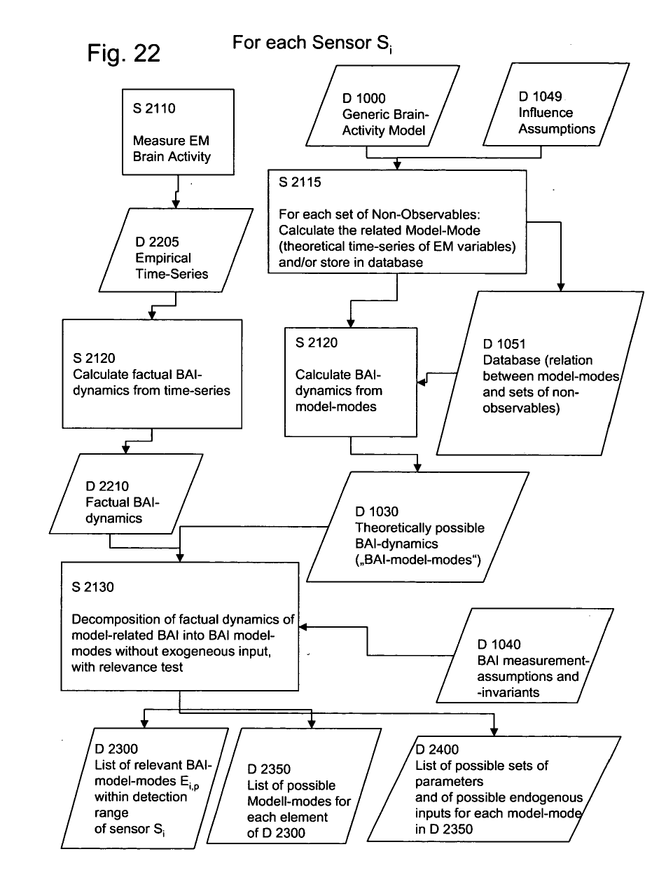

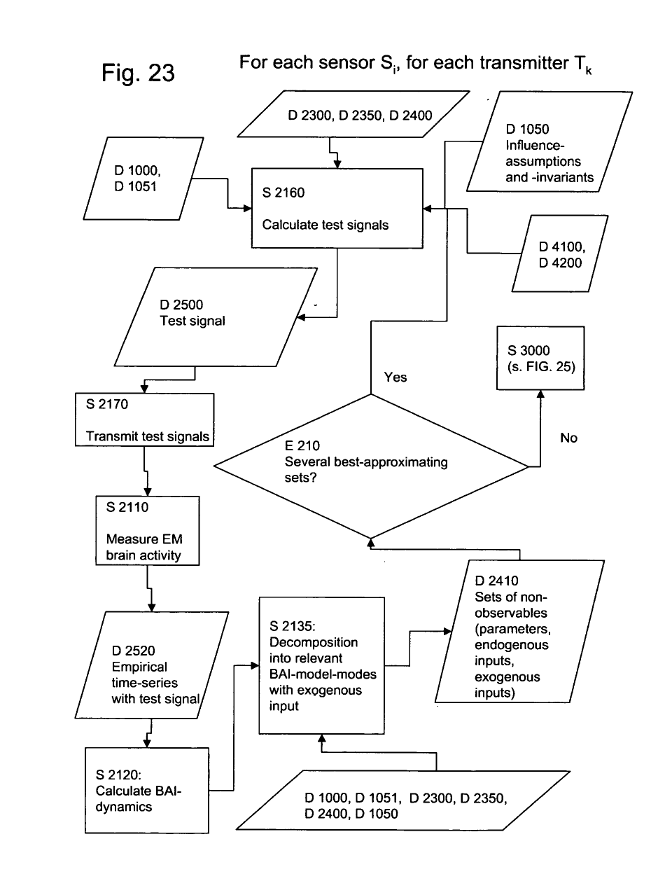

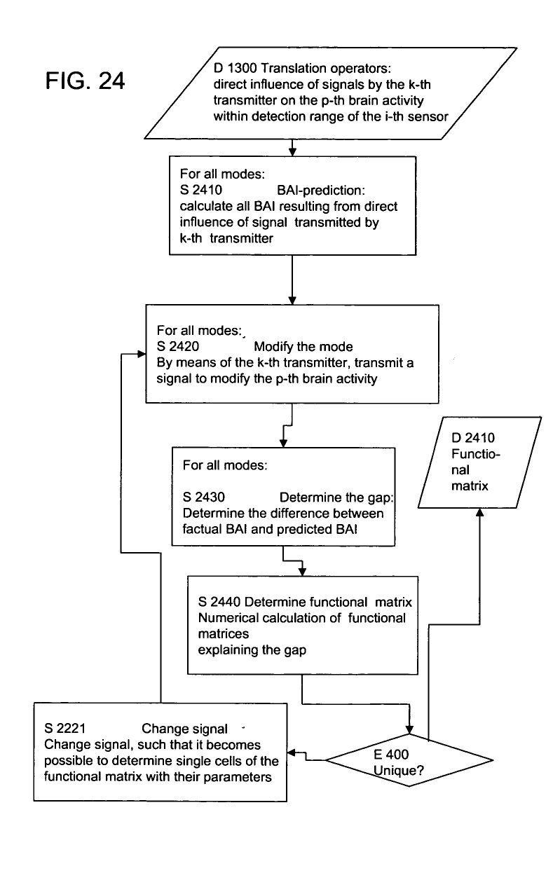

Calibration results in a specific brain model (D 1200), in which exogenous input is quantified as a control variable within the model (D 1300). Calibration is explained in more detail in FIG. 21. In the easiest case local (see FIGS. 22-23) and translocal (see FIG. 24) calibration can be distinguished from each other.

Primary target of local calibration is to determine for each sensor, for each particular user, which brain activities are occurring within a specific period of time within the detection range of each sensor (process flow shown in FIG. 22).

Secondary target of local calibration is to determine for each brain activity within the detection range of each sensor, how this brain activity can be modified by exogenous EM fields, which are generated by at least one transmitter (process flow shown in FIG. 23).

Target of translocal calibration (S 2400) is to determine type and scope of the influence of the activity of other brain constituents on the activity of the brain constituent considered.

Calibration will be explained in the following, using FIGS. 21-24:

FIG. 21 shows a part of the method, where, on the basis of data D 1100, D 1110, and D 1120 explained above, the decision (decision step E 100, explained above) is made, whether to calibrate or not.

If no calibration is required, the method continues with step S 3000 explained above. If a calibration is required, the method continues with step S 2100, which comprises local calibration (to be explained below in more detail, using FIGS. 22-23).

As local calibration calibrates brain activities locally, it is possible that the same brain activity is detected and calibrated by several sensors respectively sensor units. Therefore it is useful to identify identical brain activities as identical (step S 2300), such that brain activities calibrated several times will only be considered once, or several calibrations of the same brain activity are evaluated in a coordinated way. The respective data will be integrated into data sets D 1200 and D 1300.

In case a translocal calibration should be necessary in D 1110, this will be checked in step E 120, and, if necessary, a translocal calibration will be carried out in step S 2400 (which will be explained in more detail below, using FIG. 24). The respective data will be integrated into data sets D 1200 and D 1300.

Local calibration will now be explained, using FIGS. 22-23:

Here with each sensor brain activity is measured (S 2110). From the resulting time-series (D 2205) factual BAI dynamics will be calculated (S 2120), resulting in factual BAI dynamics (D 2210).

A brain activity model (D 1000) normally is given by differential equations, into which exogenous input is included, under certain assumptions concerning its influence (D 1049).

A MODEL-MODE is a solution of these differential equations for a certain set of non-observables, i.e. of parameters, endogenous and exogenous inputs.

These model-modes are calculated in step S 2115, and stored in a database (D 1051). This database includes a mapping from sets of non-observables to model-modes. By means of the data stored in the database D 1051 and/or using data calculated directly in step S 2115, BAI dynamics of model-modes are calculated (S 2120). This results in theoretically possible BAI dynamics (D 1030).

As BAI normally simplify model-modes, it is possible that several model-modes give similar (indistinguishable within error boundaries) BAI-model-modes. It is also possible that several sets of non-observables generate the same model-mode. This relation between BAI-model-modes, model-modes, and sets of non-observables is known from gauge theories, respectively electrodynamics (corresponding to BAI is e.g. “energy density within a volume of space”, corresponding to model-modes several electric and magnetic fields, which generate this energy density, and corresponding to the sets of non-observables different potentials, which may underlie the electric/magnetic fields). For example for stochastic modes the time series of their p-th moments are stored. Model-modes and possibly BAI-modes are stored in a data base and/or are calculable quickly. Only a finite number of non-observables are stored (lattice), likewise for model-modes and BAI-model-modes. If necessary, it is possible to refine the lattice and/or augment it (e.g. by adding new sets of parameters and/or new coefficients for endogenous and/or exogenous inputs).

It is possible, that a mode is attenuated and/or distorted and/or phase-shifted on its way from its carrier to the respective sensor (as “carrier” of a mode we denote the neuron ensemble, the activity of which generates the respective mode). The image of the mode (which arrives at the respective sensor) shall be called IMAGE MODE, analogously for the related BAI-mode.

A-priori assumptions (D 1040, FIG. 22) determine if and how an image of a BAI-model-mode is to be calculated, to become identifiable as an element of factual BAI-dynamics (S 2130). In the easiest case, the image of a BAI-model-mode is proportional to the BAI-model-mode itself, and may (apart from cases of vanishing amplitude) be set equal to the quotient of BAI-model-mode and the reciprocal of a attenuation factor. This easiest case will be assumed in the following. A (BAI-) model-mode is, in the easiest case, local and therefore describes a localized brain activity.

Decomposition according to S 2130 (see FIG. 22) includes the detection of BAI-model-modes (D 1030) within empirical BAI-dynamics, and identification of model-modes, which possibly generate these BAI-model-modes, and identification of sets of non-observables, which possibly underlie these model-modes. Here only sets of non-observables with exogenous input equal to zero are permitted. For the purpose of local calibration, endogenous input is assumed to be in the easiest case constant, in the second-easiest case to be sinusoidal, in the third-easiest case to be oscillating.

For the practical implementation of the method it is useful to restrict further analysis to relevant BAI-model-modes. A BAI-model-mode detected within factual BAI-dynamics shall be called RELEVANT, if it can be detected within factual BAI-dynamics above pre-defined thresholds for noise, measurement errors, and the like.

The decomposition results in a list of relevant BAI-model-modes (D 2300) within each detection range of each respective sensor, a list of potentially generating model-modes for each element of D 2300 (D 2350), and a list of potentially underlying sets of parameters and endogenous inputs for each model-mode in D 2350 (D 2400).

As shown in FIG. 23, for each sensor Si, and for each transmitter Tk, the following steps of the method will be carried out:

Starting from sets of parameters with endogenous inputs (D 2400), by means of testing (S 2170, transmitting test signals by means of transmitters), those sets (D 2410) of non-observables are extracted, which potentially underlie the respective model-mode under the test signal. This includes determining, with the help of the test signal, how the transmitter signal translates into exogenous input, i.e. into a part of the model equations. When this has been achieved, the influence of the exogenous signal on the respective model-mode (as well as on the BAI-model-mode) has been quantified, and can be used for the actual modification of brain activity (S 3000).

A test signal (D 2500) is, in the simplest case, resulting from possible endogenous and exogenous inputs (including: constant, oscillating, with elementary loops, i.e. time-delayed feedback of a measured signal, and many more transmitter signals).

The test signal is introduced into the generic brain model as follows: within the model equations, replace “input” by “endogenous plus exogenous input”.

Exogenous input is defined as the result of applying the translation operator to the transmitter signal.

The translation operator Ü connects the exogenous, physically measurable signal with the control variable in the equations of the specific, i.e. calibrated brain model. Depending on the parameter set and endogenous input, which underlie a respective mode, and on pattern and amplitude of the transmitter signal, different values of the control variable change the respective mode, which is physically measurable.

A test signal has the task to distinguish between different sets of parameters, endogenous inputs, and, if necessary, translation operators for the BAI-mode under consideration. A test signal (like any other transmitter signal) is characterized by an external reference value, as well as a (normally time-dependent) pattern at the locus of the transmitter, as well as a sequence of amplitude-multipliers (possibly containing only one element).

Each transmitter signal (therefore also each test signal) shall here be expressed in hybrid notation, i.e. expressed in the variables of the brain activity model, with a sequence of amplitude multipliers, and stating an external reference value, which specifies the generation of the signal.

For example an extracranial magnetic field with a field strength of 0.01 Tesla can be generated by a coil, through which a current of strength 10 is flowing. With respect to a chosen voltage unit the amplitude of the voltage, induced by changes in the magnetic field over time, is normalized to one. If the pattern of the voltage induced is sin(2*π*t*5/1000), (with voltage as variable of the brain activity model, and t time in milliseconds) under otherwise constant conditions, currents of the strength I₀, 2* I₀, 4* I₀, 8* I₀ will be stated as (0.01 T, sin(2*π*t*5/1000), (1,2,4,8)).

Furthermore, if z(t), the system behavior, is normalized to a voltage amplitude of one, feeding it back with a time delay of 10 milliseconds, with the same strength of coil currents as before, will be stated as: (0.01 T, z(t-10), (1,2, 4,8)).

The duration of partial signals (at I₀, at 2* I₀, etc.) depends on the desired effects (e.g. transients versus limit cycles), likewise possible pauses between two partial signals.

The translation from the transmitter signal to the signal at the carrier of the respective mode is subject to influence-invariants and -assumptions (D 1050).

The possible amplitude sequences are subject to technical restrictions and/or health limits (D 4100, D 4200).

In the easiest case, there are e.g. two different sets of parameters and endogenous inputs, which lead to two different model-modes, but to similar BAI-model-modes. I.e., by observing BAI, it can not be decided, which set of non-observables underlies the respective brain activity. Therefore, for a test signal pattern, a sequence of amplitude multipliers will be calculated numerically (S 2160), by inserting both sets of non-observables into the generic brain model, such that under a-priori assumptions for the translation operator (e.g. combination of phase shift with attenuation of the signal) by means of the test-signal model-modes are created, the resulting BAI-model-modes of which are different for both sets.

Normally, this numerical calculation results in a set of possible patterns for test signals, each with a set of possibly amplitude multipliers. By means of pre-defined criteria (e.g. minimal perturbation of all occurring brain activities with the exception of the brain activity to be tested), an optimal test signal will be selected.

Starting with transmitting the signal (S 2170) with the first amplitude in the sequence, and measuring electromagnetic brain activity (S 2110) minimally with the sensor considered (Si), maximally with all sensors of the sensor grid (see e.g. FIG. 5), results in a time series (D 2520), from which BAI-dynamics are calculated (S 2120).

Subsequently factual BAI dynamics are decomposed into BAI-model-modes with exogenous input (S 2135). For this decomposition only sets of parameters and exogenous inputs from D 2400 (see FIG. 22) are admitted, whereby the setting “exogenous input equal to zero” is replaced by a possibly different exogenous input, the pattern of which results from influence-invariants and -assumptions, and the strength of which results from the (normally non-linear) changes related to different amplitudes of an amplitude sequence.

If this decomposition results in several best-approximating sets of non-observables (E 210), another test signal is to be calculated, and the loop following S 2160 will be run again, with, at S 2135, consideration of D 2410 (s. FIG. 23) instead of the former set of parameters and endogenous inputs from D 2400. In case several repetitions of this loop fail to generate results, the orientation of the transmitter needs to be changed, or another transmitter shall be selected.

In summary, after having completed local calibration it is known,
which model-modes are in the detection range of the sensor,
which parameter set, and which endogenous input generates the respective model-mode, and
how to influence the respective model-mode.

In addition to local calibration it is possible, in step S 2300, to identify identical carriers of model modes.

If Aₖₗ is a mode belonging to sensor Sₖ, characterized by parameters Pₖₗ, endogenous input Iₖₗ(t), and translation operators Ü_{kl,x}(t), and if Aₘₙ (t) is a mode belonging to sensor Sₘ, characterized by parameters Pₘₙ, endogenous Input Iₘₙ (t) and translation operators Ü_{mn, x} (t) (X enumeration of transmitters), then the following statement is valid: if each Pₖₗ is equal to each respective Pₘₙ, and each Iₖₗ (t) equals the respective Iₘₙ(t), then both modes are identical. These identical modes do not have the same carrier, if there is a transmitter T_{X}, such that Ü_{kl,x} (t)unequal to Ü_{kn, x} (t) and/or Ü_{ml, x} (t) unequal to Ü_{mm, x} (t).

Procedural step S 2300 is not essential, but results in a possible minimization of the functional matrix (D 2410, see FIG. 24) in the following translocal calibration.

With translocal calibration (S 2400) type and scope of the influence of other modes and/or of sensory input on a mode will be determined (indirect exogenous influence, i.e. presentation of visual, acoustic, tactile, or other sensory stimuli, is mediated by the sensory system, results in delays, and is modeled as part of endogenous input). Tool for this purpose is the FUNCTIONAL MATRIX (D 2410): if in total n different model modes have been found within the detection range of at least one sensor, this functional matrix is a n*n matrix, the (i,j)-cell of which shows an entry, if an influence of mode i on mode j can be determined. The entry quantifies this influence, such that in the equations of the brain model for the mode j “endogenous input” is replaced by “possibly time-delayed function of the i-th mode plus further endogenous input”.

The method for filling in required parts of the functional matrix is shown in FIG. 24. Knowing the functional matrix or parts thereof allows for the calculation of phenomena of the respective brain model, which include several constituents of the model (e.g. synchronization, phase-locking, and many more), by virtue of conventional numerical methods, neural networks, nonlinear dynamics, and others.

Translation operators (D 1300) obtained from local calibration, as well as further non-observables allow for BAI predictions (S 2410) for all modes, which take translocal influences into account in aggregated way only. When actively altering a mode (S 2420), a possible difference between factual BAI and predicted BAI might become apparent. This difference is captured in step S 2430. These differences are essentially based on translocal influences. In step S 2440 such functional matrices are calculated, which are (when included into the equations of the brain model) capable of explaining the above difference.

In E 400 it is tested, whether the mapping from the above difference to functional matrices is unique. If not, in step S 2221 the test signal is modified, in order to narrow the set of potentially suitable functional matrices. This iteration will be stopped after having uniquely determined the desired elements of the functional matrix.

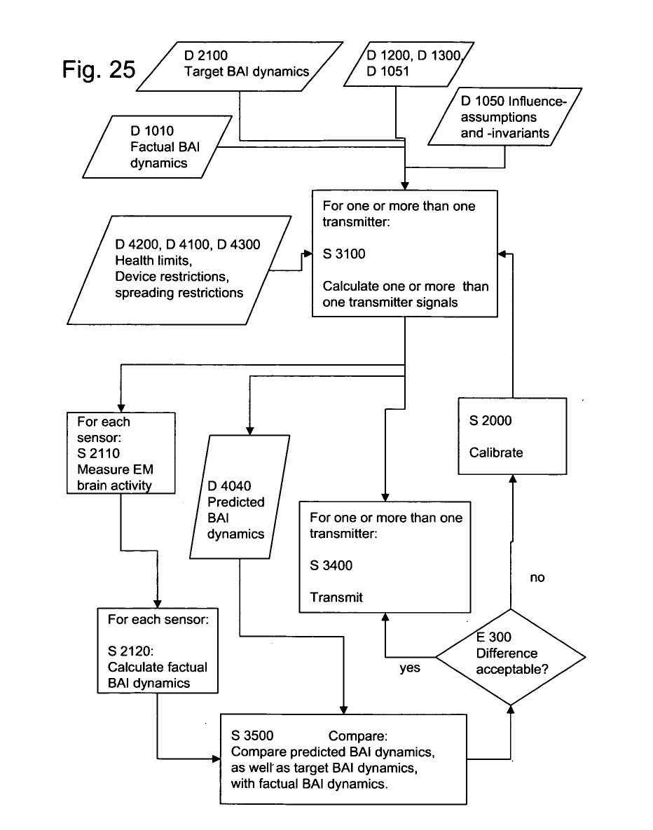

Following calibration, the preferably EM controlling or regulating (S 3000) of brain activity by brain-activity-model-based feedback loops is targeted at achieving and sustaining BAI-target dynamics. This is shown in detail in FIG. 25.

Here, at first, transmitter signals are calculated, which are theoretically suitable to transform factual BAI dynamics (D 1010) into target BAI-dynamics (S 3100). In a preferred embodiment, these transmitter signals are composed from elementary signals the impact of which is calculable within the specific brain model, respectively known from calibration. For this composition, multiple influences (influences of signals of one transmitter on several brain activities), spatial composition (signals of several transmitters), and composition in time (sequence of signals) are suitable. After a feasibility test of the signals calculated (with respect to health limits (D 4200), device restrictions (D 4100), possibly spreading restrictions (D 4300)), by means of a utility function the best transmitter signal will be selected and transmitted (S 3400). The theoretical impact of a signal is known within a specific brain model, and allows for prediction of BAI dynamics (D 4040). If (during ongoing EM measurements and ongoing factual-BAI calculations) the comparison of factual- and target-BAI-dynamics (S 3500) should result in substantial differences (E 300), S 3000 will be interrupted on a provisionary basis, to improve the calibration with a further calibration step (S 2000). In this case the method subsequently proceeds to step S 3100, to again calculate the transmitter signals.

The main steps of the method shall now be explained, using some examples. The examples will be explained using walkthroughs of the flow diagrams (FIGS. 20-25):

### EXAMPLE 1

FIG. 20:

D 2000 (Behavioral Target):

Amplification of positive emotions for the duration of the application

D 3000 (Behavioral Model):

The following correspondence is called “reduced Davidson-model” (see e.g. [11]): positive emotions correspond to a higher quotient of beta to alpha activity in the left-frontal cortex (beta=13-30 Hz, alpha=8-12 Hz).

S 500 (BAI Specification and Calculation, Specification of Target BAI-Dynamics):

The Davidson-model is based on the power-spectrum of EEG-signals. A suitable BAI is therefore a sequence of squared moduli of Fourier coefficients, to be calculated via Fast Fourier Transformation, for example for frequencies between 1 and 50 Hz. Derived BAI is

$$Pos \text{:} = {\sum }_{i = 13}^{30} {f}_{i}^{2} / {\sum }_{i = 8}^{12} {f}_{i}^{2}$$

(Assumption: only integer frequencies, Fourier-window e.g. 500 milliseconds, fᵢ modulus of the Fourier coefficient of the i-Hertz-mode. To cover the frequencies from 1 to 50 Hz, according to Nyquist at least 100 data points are necessary). BAI(100) is the power spectrum of the data points 1-100, BAI(101) the power spectrum of the data points 2-101, etc. Pos(100) is derived from BAI(100), Pos(101) from BAI(101), etc. This derived BAI shall be increased, e.g.: from a time t on (or from the respective data point on), Pos(t, with influence)>2*Pos(t, without influence). This requirement determines all target BAI dynamics.

D 4000 (Minimal Device Requirements):

Surface-EEG with one electrode left-frontal, and a reference-electrode, e.g. at the ear (see [16]), one extracranial coil (as transmitter) left-frontal. At 100 data points per half second (see S 500) there is a minimal sampling rate of 200/sec.

D 2100 (Target BAI-Dynamics):

Pos(t, with influence)>2*Pos(t, without influence)

D 2200 (BAT Calculation Rules):

Using Fast Fourier Transformation, squared moduli (fᵢ)² of Fourier coefficients of each i-th mode (i between 1 and 50 Hz) are calculated.

$$Pos \text{:} = {\sum }_{i = 13}^{30} {f}_{i}^{2} / {\sum }_{i = 8}^{12} {f}_{i}^{2}$$

D 1000 (Generic Brain Activity Model):

Simplified Wilson-Cowan-model (derived from [1], with refractory times set to zero, likewise for transmission delays, and many more), where brain activity is based on the activity of neural oscillators. A neural oscillator oscillates or does not oscillate, depending on its input, and on also non-observable physiological parameters. A neural oscillator is an ensemble of interconnected excitatory and inhibitory single neurons (in [1] of the order of 105 single neurons. Here also multiples of this number are admitted, as long as the respective ensemble of neurons obeys the following equations), modeled by a system of two nonlinear differential equations:
τ\dot{x}=−x+S(ax−by+ρₓ)
τ\dot{y}=−y+S(cx−dy+ρ_{y})

Here S(ξ):=1/(1+exp(5−ξ)) is a sigmoid function.

“x”, “y” are electromagnetic variables, observable as αx+βy, whereby α, β are both from the interval [−1,1] , and are called MIXING ANGLES. “x” represents the set of excitatory single neurons, “y” represents the set of inhibitory single neurons (“set”=all such neurons within the neural oscillator).

“a”, “b”, “c”, “d” are slow non-observables.

“a” models the influence of the set of excitatory single neurons on itself,

“d” models the influence of the set of inhibitory single neurons on itself,

“b” models the influence of the set of inhibitory single neurons on the set of excitatory single neurons,

“c” models the influence of the set of excitatory single neurons on the set of inhibitory single neurons.

“ρₓ” models endogenous input from outside the neural oscillator into the set of excitatory single neurons,

“ρ_{y}” models endogenous input from outside the neural oscillator into the set of inhibitory single neurons.

Within the model, input variables are not necessarily considered as slow (this aspect will be given particular consideration when explaining translocal calibration). All times are expressed in milliseconds, all other units are dimensionless. “τ” on the left hand side of the Wilson-Cowan equation models the membrane time constant in milliseconds, another non-observable.

To include exogenous influence, the simplified Wilson-Cowan-model will be augmented: direct electromagnetic exogenous influence is modeled as part of input: ρₓ=ρ_{x,endo}+ρ_{x,exo} and ρ_{y}=ρ_{y,endo}+ρ_{y,exo}, where ρₑₓₒ=Üᵢ (transmitter signal) denotes exogenous input transmitted from the i-th transmitter into the neural oscillator under consideration, and “endo” stands for “endogenous”. Ü is a translation operator, which depends, amongst other factors, on the distance of the neural oscillator considered from the respective transmitter, on the orientation of the single neurons with respect to the transmitter axis, on other physiological parameters, as well as on the type of signal transmitted. Translation operators are non-observables.

It is known, that the above non-observables vary by factors of up to 100 between different people, even may vary considerably within different brain areas of one brain in vivo, and may also change (slowly compared to EM oscillators). The resulting plurality of theoretical and factual modes contains an almost arbitrary number of modes. Controlled local modification without local calibration is therefore normally not possible. Under the assumption of relatively constant endogenous input translocal calibration is not necessary.

S 800 (Specification of Minimum Calibration Requirements):

τ, a, b, c, d, ρₓ, ρ_{y}, translation operators for one sensor-transmitter-pair. Only local calibration is necessary.

D 1100 (Minimal Calibration Requirements):

Locally calibrate τ, a, b, c, d, ρₓ ρ_{y}, translation operators for one sensor-transmitter-pair.

E 100 (Calibration Necessary?):

Local calibration is necessary.

D 4100 (Device Restrictions):

Depend on the device used, e.g. on the input-side conventional self-adhesive electrodes, digitalized with a sampling rate of 200/sec, on the output side e.g. a coil with a maximal magnetic field of 0.5 Tesla. The restriction to only one transmitter results in the impossibility of influencing several modes independently.

D 4200 (Health Limits for Exogenous Electromagnetic Fields):

Includes e.g. the recommendations of [14].

S 2000 (Calibration):

Will be explained in detail when walking through the flow diagrams of FIGS. 21-24.

D 1200 (Specific Brain Activity Model):

D 1000 with empirically determined τ, a, b, c, d, ρ_{x,endo} ρ_{y,endo}, as well as D 1300 (for each relevant brain activity of each user within the time frame considered).

D 1300 (Quantification of the Influence of Exogenous EM Fields on Brain Activity):

Empirically determined influence of exogenous EM fields on each relevant brain activity of the respective user within the time frame considered, represented by control variables ρ_{x,exo} and ρ_{y,exo} in the simplified Wilson-Cowan equations modeling this brain activity.

S 3000 (Control or Regulation of Brain Activity by Means of Targeted Modification of Control Variables and Measurement/Calculation of BAI):

Will be explained in detail when walking through the respective flow diagram (FIG. 25).

FIG. 21:

D 1100 (Minimal Calibration Requirements):

As before.

D 1110 (Calibration Type):

Local.

D 1120 (Range of Calibration):

Sensor S₁, transmitter T₁.

E 100 (Calibration?):

Yes.

S 2100 (Local Calibration):

Will be explained in detail when walking through the flow diagrams of FIGS. 22-23. In this example, there is only one sensor-transmitter-pair, which needs to be calibrated.

S 2300 (Identification of Identical Brain Activities):

Skipped, because multiple sensors and transmitters are not available.

E 120 (Translocal Calibration?):

No.

D 1200 (Specific Brain Activity Model):

As before.

D 1300 (Quantification of the Influence of Exogenous EM Fields on Brain Activity):

As before.

S 3000:

See FIG. 25.

FIG. 22:

2110 (Measuring EM Brain Activity with S₁):

By virtue of the left-frontal electrode and the reference electrode differences in potential will be measured, and digitalized with 100 values per second. The time series generated (D 2205) is stored.

D 2205 (Empirical Time Series for S₁):

The time series of differences in electric potential, resulting from S 2110.

S 2120 (Calculation of Factual BAI Dynamics from the Time Series for S₁):

With a sampling rate of 100/second frequencies between 1 and 50 Hertz are obtained. By virtue of FFT (Fast Fourier Transformation), from a data window of given length (100 data points) the power spectrum is calculated. From there, Pos is calculated in accordance with the formula in S 500. (S 2130 already starts with BAI(100).)

D 2210 (Factual BAI Dynamics for S₁):

Factual dynamics of the power spectrum, i.e. BAI(100), BAI(101), BAI(102), etc. Additionally factual dynamics of Pos, i.e. Pos(100), Pos(101), Pos(102), etc.

D 1000 (Generic Brain Model):

As before.

D 1049 (Influence Assumptions):

Explained in detail at D 1050, FIG. 23.

D 1051 (Database):

The infinite plurality of possible parameter combinations in the generic model is, a priori, reduced to a finite one: firstly by calculating the limits of the parameter range, for which endogenous inputs exist, which lead to a non-constant EM output, secondly by digitizing an n-fold bigger superset of this range (defining a parameter lattice, n natural number). Each parameter set is contained in this lattice. Each endogenous input is here, in the simplest case, considered to be constant. As initial values for the activity variables x and y in the simplified Wilson-Cowan-equations, random variables (uniformly distributed in the interval [0,1]) are chosen.

Now it shall be presented with an example, with, at first, exogenous input set to zero, which consequences for the power spectrum of the resulting mode different parameter values have, for constant endogenous input (x-axis shows frequency in Hertz, y-axis shows squared modulus of the respective Fourier coefficient).

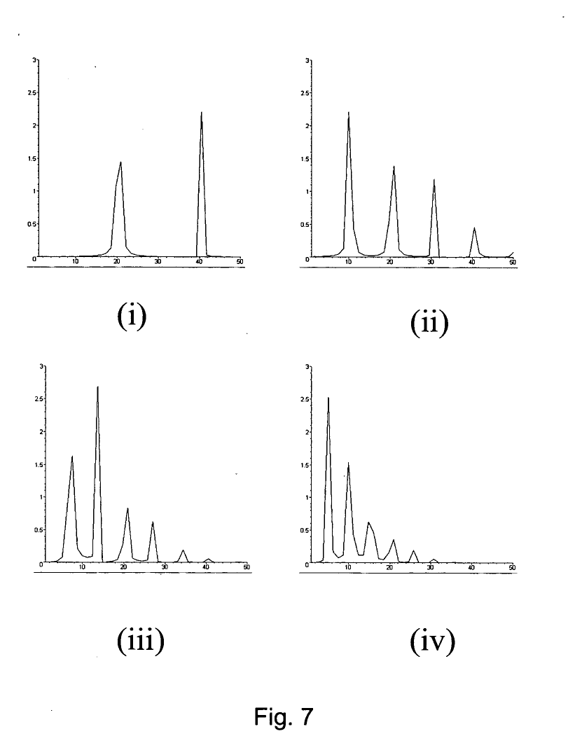

FIG. 7 shows BAI modes under variation of the membrane time constant τ:
(i) is generated by τ=5, a=22, b=20, c=14, d=3, at ρₓ=1.5 and ρ_{y}=0.
(ii) is generated by τ=10, a=22, b=20, c=14, d=3, at ρₓ=1.5 and ρ_{y}=0.
(iii) is generated by τ=15, a=22, b=20, c=14, d=3, at ρₓ=1.5 and ρ_{y}=0.
(iv) is generated by τ=20, a=22, b=20, c=14, d=3, at ρₓ=1.5 and ρ_{y}=0.

These modes are stationary under constant input, therefore the BAI shown, displayed over time, are also BAI dynamics. Transients between some initial values and the subsequent oscillations, which only have a few milliseconds duration, are always omitted.

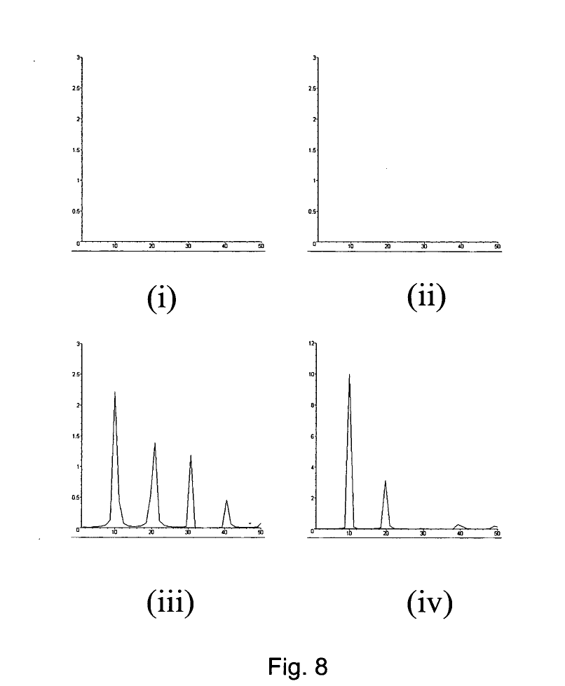

FIG. 8 shows the BAI of modes under variation of the self-excitatory parameter a:
(i) is generated by τ=10, a=14, b=20, c=14, d=3, at ρₓ=1.5 and ρ_{y}=0.
(ii) is generated by τ=10, a=18, b=20, c=14, d=3, at ρₓ=1.5 and ρ_{y}=0.
(iii) is generated by τ=10, a=22, b=20, c=14, d=3, at ρₓ=1.5 and ρ_{y}=0.
(iv) is generated by τ=10, a=26, b=20, c=14, d=3, at τₓ=1.5 and ρ_{y}=0.

For the two smaller self-excitatory parameters the lack of oscillations is clearly noticeable.

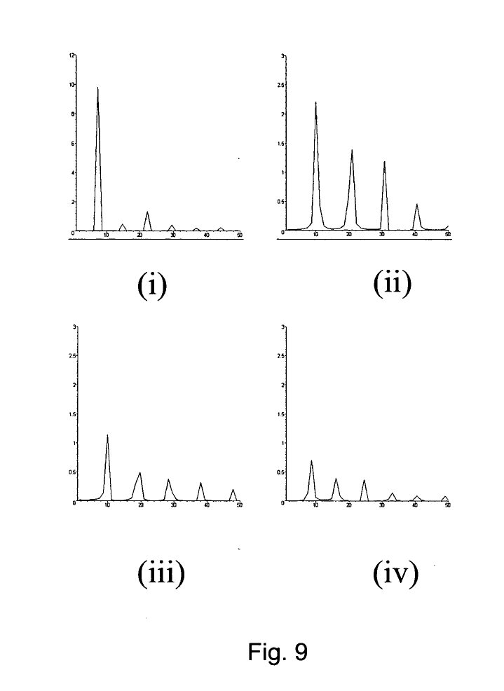

FIG. 9 shows the BAI of modes under variation of the cross-inhibitory parameter b:
(i) is generated by τ=10, a=22, b=15, c=14, d=3, at ρₓ=1.5 and ρ_{y}=0.
(ii) is generated by τ=10, a=22, b=20, c=14, d=3, at ρₓ=1.5 and ρ_{y}=0.
(iii) is generated by τ=0, a=22, b=25, c=14, d=3, at ρₓ=1.5 and ρ_{y}=0.
(iv) is generated by τ=10, a=22, b=30, c=14, d=3, at ρₓ=1.5 and ρ_{y}=0.

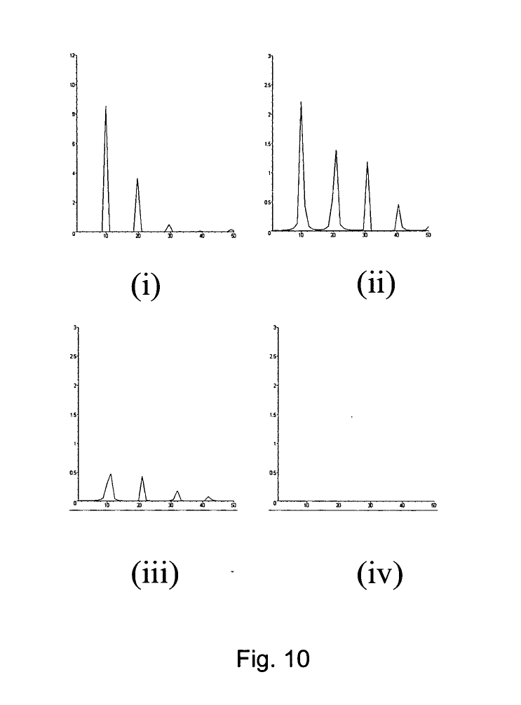

FIG. 10 shows the BAI of modes under variation of the cross-excitatory parameter c:
(i) is generated by τ=10, a=22, b=20, c=10, d=3, at ρₓ=1.5 and ρ_{y}=0.
(ii) is generated by τ=10, a=22, b=20, c=14, d=3, at ρₓ=1.5 and ρ_{y}=0.
(iii) is generated by τ=10, a=22, b=20, c=18, d=3, at ρₓ=1.5 and ρ_{y}=0.
(iv) is generated by τ=10, a=22, b=20, c=22, d=3, at ρₓ=1.5 and ρ_{y}=0.

Lack of oscillations in case of strong cross-excitation of inhibitory neurons within the ensemble is clearly noticeable (case iv).

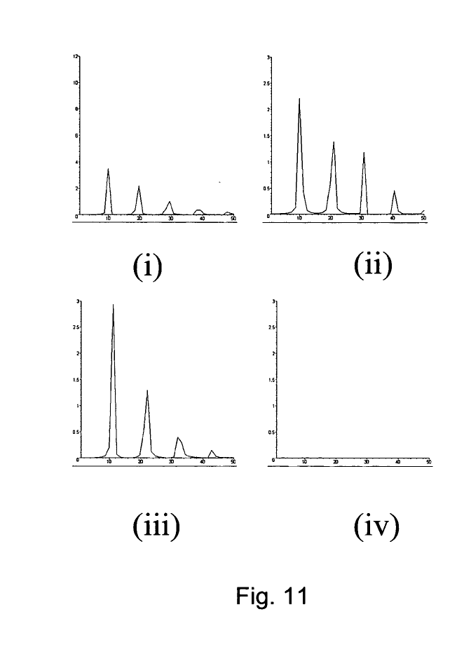

FIG. 11 shows the BAI of modes under variation of the cross-inhibitory parameter d:
(i) is generated by τ=10, a=22, b=20, c=14, d=0, at ρₓ=1.5 and ρ_{y}=0.
(ii) is generated by τ=10, a=22, b=20, c=14, d=3, at ρₓ=1.5 and ρ_{y}=0.
(iii) is generated by τ=10, a=22, b=20, c=14, d=6, at ρₓ=1.5 and ρ_{y}=0.
(iv) is generated by τ=10, a=22, b=20, c=14, d=9, at ρₓ=1.5 and ρ_{y}=0.

Lack of oscillations in case of strong self-inhibition is clearly noticeable (case iv).

S 2115 (For Each Set of Non-Observables: Calculation of the Related Model-Mode and/or Retrieval of the Mode from the Data-Base):

Inserting non-observables into the simplified Wilson-Cowan equations and numerically solving these.

S 2120 (Calculation of BAI Dynamics from Model-Modes and/or Retrieval from Data-Base):

Applying BAI-calculation to the results of S 2115.

D 1030 (BAI Model-Modes):

Results of S 2120, partially represented under D 1051.

D 1040 (BAI Measurement-Invariants and -Assumptions):

For the purpose of detection of model-modes in factual modes, in the easiest case BAI are chosen, which are partially or completely invariant with respect to the measurement method, or such, for which such invariance can be assumed. In the example given (power spectrum) this assumption is: the attenuation of the power spectrum between the carrier of a model-mode and the sensor is frequency-independent. This means, the quotients of coefficients of a model-mode stay invariant on its way to the sensor. Furthermore, the signal is assumed to be piecewise stationary (duration of stationarity longer, on average, than the size of the Fourier window). Furthermore it is assumed (s. [1]), that the differences in electric potential emanating from excitatory and inhibitory neurons involved in the respective brain activity enter the EEG signal with equal weights, as x-y, i.e. the mixing angles are α=1, β=−1.

S 2130 (For S₁: Decomposition of Factual BAI Dynamics of Model-Based BAI-Modes into BAI-Model-Modes, with Relevance Testing):

In principle, model-based BAI-modes are per default EM variables, which are described in the brain model (e.g. differences in potential). In the example given (quasi-stationary neural oscillators), the power spectrum is used instead. A-priori changes of the default settings in accordance with the brain model used are left to one skilled in the art. Here: Identify a model-mode, the BAI-model-mode of which (in the easiest case divided by an attenuation factor) best explains (with respect to a norm, e.g. L1) the power spectrum. Remove this mode divided by the attenuation factor from the power spectrum. Identify a model-mode, the BAI-model-mode of which best explains (with respect to the norm) the residual power spectrum, etc., until a pre-defined threshold is passed from above.

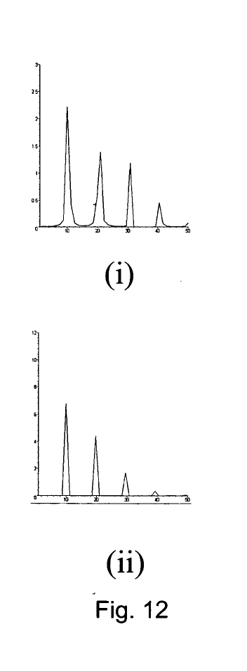

If, e.g., the factual power spectrum modulo noise has stationary maxima at 10 Hz (with a modulus of 10), at 20 Hz (with a modulus of 5), and smaller maxima at further multiples of 10 Hz, in first approximation (i.e. with respect to maxima of peaks instead of area under the peaks) both BAI model-modes shown in FIG. 12 explain the factual power spectrum equally well. The first one (E_{1,1}) has a attenuation of ca. 10/2.2, the second one (E_{1,2}) has a attenuation of ca. 10/6.8. Removing e.g. the first BAI-model-mode times 0.22 results approximately in the zero power spectrum. Analogously for the second BAI-model-mode times 0.68. This means, in the example given, only the model-modes E_{1,1,} E_{1,2} have been identified, both for sensor S₁. It is not possible to distinguish between these modes in case of unchanged endogenous input. Therefore the distinction is carried out by means of test signals.

D 2300 (List of Relevant BAI Model Modes within the Detection Range of Each Sensor S₁):

Here, the list consists only of E_{1,1} and E_{1,2,} both for sensor S₁.

D 2350 (List of Possible Model Modes for Each Element of D 2300):

This list contains, amongst other model modes, the model modes M_{1,1,1} (for E_{1,1}) and M_{1,2,1} (for E_{1,2}), the generation of which will be considered in the following.

D 2400 (List of Possible Parameter Sets with Endogenous Input for Each Model Mode in D 2350):

This list contains, amongst other parameter sets with endogenous input, for each model mode from D 2350 parameter sets with endogenous input, which generate this mode according to the simplified Wilson-Cowan equations. D 2400 here e.g. comprises for M_{1,1,1} the set of parameters τ=10, a=22, b=20, c=14, d=3, with endogenous inputs ρₓ=1.5 und ρ_{y}=0, and for M_{1,2,1} the set of parameters τ=10, a=26, b=20, c=18, d=3 with endogenous inputs ρₓ=1.36 and ρᵥ=−0.14.

FIG. 23:

D 2300, D 2350, D 2400, D 1000, D 1051, D 4100, D 4200:

As before.

D 1050 (Influence-Invariants and -Assumptions):

The control variables in the equations of a generic brain model are produced by applying (to each model mode and each transmitter T₁ used) an a-priori unknown translation operator to the signal transmitted. In the example given:
τ\dot{x}=−x+S(ax−by+ρ_{x,endo}+Üₓ (TransmitterSignal))
τ\dot{y}=−y+S(cx−dy+ρ_{y,endo}+Ü_{y} (TransmitterSignal))

Concerning the shape of the translation operator, testable assumptions are made, for example for the special case of acoustic stimulation Üx=Üy (see [18]). The same assumption is made here, furthermore the assumption, that on average, within a range of validity, two transmitter signals which differ only by their amplitudes (e.g. amplitude relation 1:\dot{r}, r real number >0) will translate into exogenous inputs, the amplitudes of which also have a 1:r relation. Furthermore, it is assumed, that ρ_{x,endo}, ρ_{y,endo} are stationary during local calibration.

S 2160, D 2500:

As a test signal, in principle every signal is suitable, which is in accordance with device restrictions (D 41 00) and health limits (D 4200).

It is the purpose of the test signal, to firstly eliminate candidates for generating parameters and endogenous input from D 4200 until the mapping from observed factual BAI dynamics becomes unambiguous, and secondly to determine the translation from test signal to exogenous input.

From the set of all possible test signals, test signals, which fulfill these requirements, can be determined numerically (e.g. by selecting an ordered base of functions plus brute-force-calculations).

It is a suitable simplification, to require from a test signal a relation to the brain activities measured, as well as to the brain activity model used, as well as (in a self-consistent way) to a signal which might be used for modification (S 3000). For linear oscillators, frequencies commensurate and incommensurate to the oscillations frequency are to be distinguished. In the example given, the basic frequency (smallest frequency in the power spectrum, which is not generated by noise) is used analogously: According to FIG. 12 the basic frequency of the two similar BAI model modes is 10 Hz. Therefore incommensurate test signals can be sinusoid signals with a frequency of 3 Hz, 7 Hz, 9 Hz, 11 Hz etc. Commensurate test signals can be sinusoid signals with a frequency of 2 Hz, 4 Hz, 5 Hz, 6 Hz, 8 Hz, 10 Hz etc. (Phase shifts are irrelevant for the example given, with one mode on one carrier). Furthermore elementary loops can, in principle, be used as test signals, i.e. time-delayed feedback of a measured signal, e.g. of the shape
exo(t)=x(t−10)=y (t−10).

In addition to the possibilities shown concerning the pattern of the signal, different amplitude multipliers are useable, starting with low amplitudes. Example: test signal=(0.05 Tesla, sin(2*π*t*3/1000), (1,2,3,4)), t time in milliseconds.

S 2170 (Transmitting the Test Signal):

Performed in the order of signals determined in D 2500, with pauses between two transmissions of the order of multiples of the membrane time constant, to avoid temporal summation effects.

S 2110, D 2510:

As before. If measurement and transmission of the test signal are carried out at the some time, and e.g. missing or incomplete shielding of the sensor(s) leads to artifacts, these are removed from the signal by calculation. Calculating the strength of an artifact, which a transmitter creates in a sensor normally takes into consideration the distance between both, and constitutive relations of the substances between both. Before starting the calibration, it is possible to measure these artifacts directly, by means of transmitting very-low-amplitude signals, which can be well-distinguished from natural activity (of such low amplitude that the possibility of thereby influencing neural activity can be assumed as unlikely). Artifacts, which are possible without transmitter signals, (e.g. by muscle movements during the measurement) are removed by conventional means.

S 2120:

As before.

S 2135 (Decomposition with Respect to Relevant BAI Model Modes with Test Signal):

In contrast to S 2130 here the decomposition is not performed with respect to arbitrary BAI model modes, but with respect to BAI model modes with test signal.

Here only parameter sets and endogenous inputs from D 2400 are admitted, which now, by virtue of exogenous input, may lead to other solutions (model modes with test signal), and therefore to other BAI-model modes with test signal.

The attenuation of S 2130 is maintained, under the assumption, that the locus of the carrier of the respective mode does not change under exogenous input.

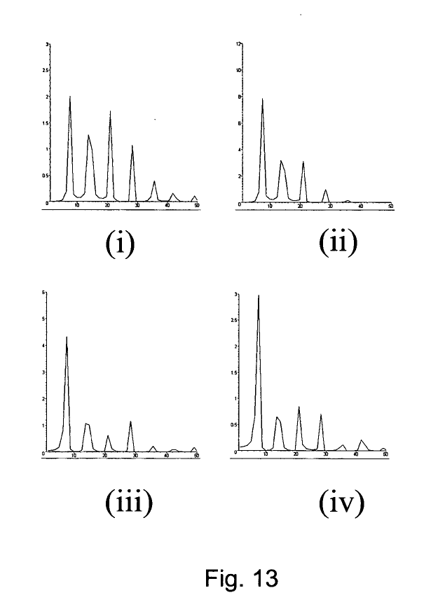

BAT model modes to be considered have e.g. under sinusoid exogenous sine input of frequency 7 Hz for mode I (see FIG. 12(i)), for multipliers 2, 4, 6, 8 the shape shown in FIG. 13:
(i) for ρₑₓₒ=2*sin(2πt*7/1000)
(ii) for ρₑₓₒ=4*sin(2πt*7/1000)
(iii) for ρₑₓₒ=6*sin(2πt*7/100)
(iv) for ρₑₓₒ=8*sin(2πt*7/1000)

Here already some observations, concerning the quantification of the influence of the signal, result: The power spectrum, which is inverted with respect to the 7 Hz and the 14 Hz component, normalizes when the multiplier is doubled from 2 to 4. The squared modulus of the 7 Hz component shows, when increasing the multiplier from 2 to 4 the following approximate ratio: ¼, from 4 to 6: 3/2, from 6 to 8: 4/3.

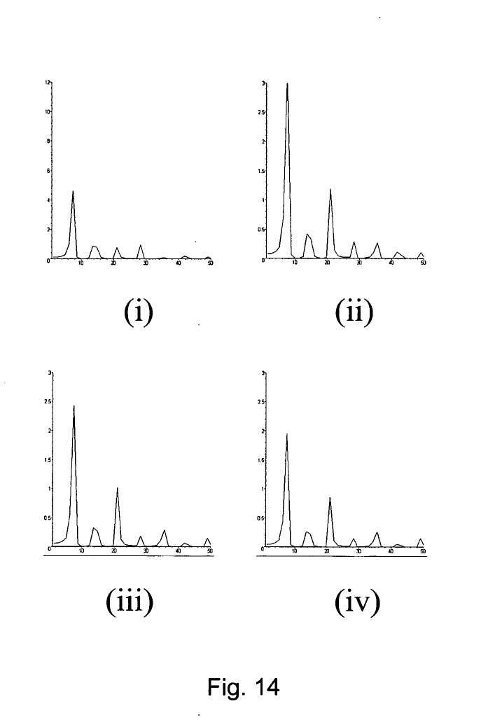

The same exogenous input generates, for the other BAT model mode (mode II, see FIG. 12 (ii)), the power spectra shown in FIG. 14 for multipliers 2, 4, 6, 8:
(i) for ρₑₓₒ=2*sin(2πt*7/1000)
(ii) for ρₑₓₒ=4*sin(2πt*7/1000)
(iii) for ρₑₓₒ=6*sin(2πt*7/1000)
(iv) for ρₑₓₒ=8*sin(2πt*7/1000)

Even at small multipliers the test signal discriminates between mode I and mode II, as visible from a comparison of FIG. 13(i) and FIG. 14(i).

The multiplication of the signal intensity I, allows for influence calibration: If e.g. in the case of mode II doubling the signal intensity I₁ to I₂ decreases the ratio of the first peak divided by the second peak from 6 to 2.5 Checking this result with the BAI model mode data base will show, that I1 corresponds to a multiplier of 2 (FIG. 14(i)), and I₂ to a multiplier of 4 (FIG. 14(ii)).

The previous example ignored (for reasons of simplicity of presentation) the—in reality—rather frequent complication, that measurement and transmission might not be carried out at the same time. In case of measurement interruptions which are small compared to the oscillation period, the interrupted measured signal can normally be reconstructed using conventional signal theoretic methods. In the following we will consider an uninterrupted measured signal as given. Transmission interruptions will be presented for the case of elementary loops with exogenous input.

As an example for an elementary loop, the measured signal x(t)-y(t) will be fed back into the system with a time delay τ, i.e. for τ=10 milliseconds we have the equations:
τ\dot{x}=−x+S(ax−by+ρ_{x,endo}+ρ_{x,exo})
and
τ\dot{y}=−y+S(cx−dy+ρ_{y,endo}+ρ_{y,exo})
where
ρ_{x,exo}=ρ_{y,exo}=μ*(x(t−10)−y(t−10)), if one assumes that the translation operator does not distort and/or further delay the signal. μ is a multiplier to be determined by successive amplitude doubling of the transmitter signal. All other symbols have already been explained.

For the case of not simultaneously active sensors and transmitters (relevant e.g. for the sensor being blocked during the transmission), the switching time is chosen as the delay time. In the previous example, every 10 milliseconds one switches between measuring and transmitting. In the above equations
ρ_{x,exo}=ρ_{y,exo}=μ*(xi(t))
with xi (t):=if(t mod 20>10, x(t−10)−y (t−10), 0), in generic code.

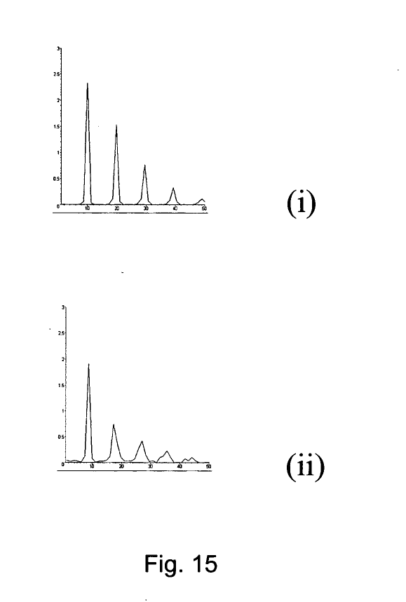

FIG. 15 shows mode I with this exogenous input for μ=4 (i) and μ=8 (ii).

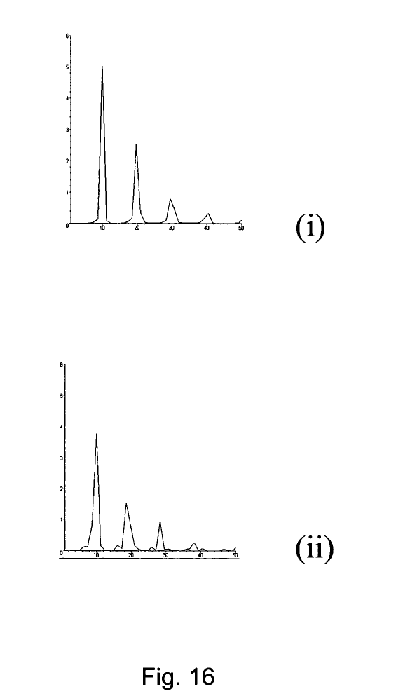

FIG. 16 shows mode II with this exogenous input for μ=4 (i) and μ=8 (ii).

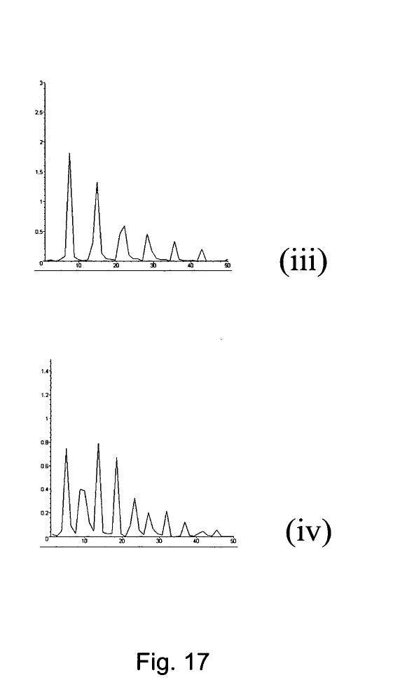

FIG. 17 shows mode I with this exogenous input for μ=12 (iii) and μ=16 (iv).

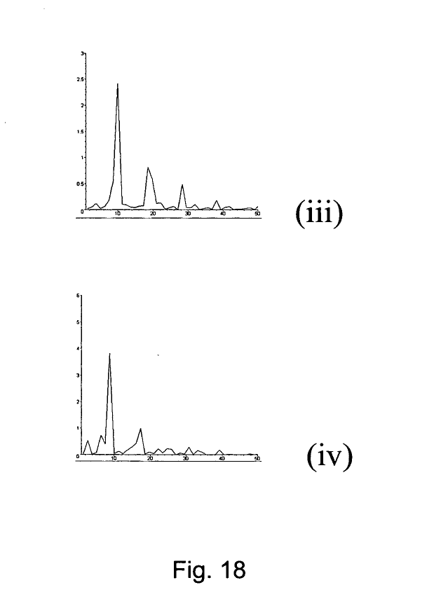

FIG. 18 shows mode II with this exogenous input for μ=12 (iii) and μ=16 (iv).

Here the test signal also discriminates between modes I and II, e.g. by means of frequency shift (FIG. 15(ii) versus FIG. 16(ii)) and/or the ratio of maxima of consecutive peaks (FIG. 17(iii) versus FIG. 18(iii)). The quotient of maxima of consecutive peaks of a mode, for different endogenous inputs, allows here, analogously to the presented case of sinusoid input, for calibration of the influence of exogenous input.

There are no considerations of phase in this simple example. For a fine-tuning-calibration additional sequences of phase-shifted test signals are used.

For the further presentation of example I it is assumed, that by means of time-delayed feedback, test signals it was determined, that the brain activity observed is generated by mode I (with non-observables τ=10, a=22, b=20, c=14, d=3, with ρ_{x,endo}=1.5 and τ_{y,endo}=0), into which the time-delayed feedback signal of transmitter T₁ enters as 40* [magnetic field strength in Tesla] *xi (t). Furthermore it is assumed that the mode on its way from its carrier to the sensor S₁ is attenuated by a factor of 10/2.2.

Remark 1: It general it can not always be assumed that suitable modes are generated by parameters and inputs of the lattice chosen, i.e. local refinements and/or interpolations will be necessary.

Remark 2: Results, which are inconsistent within a signal sequence indicate, that during transmission one or more jumps of the quasi-stationary endogenous input have occurred. In this case modes with time-dependent input need to be considered for calibration.

E 210 (Several Best-Approximating Sets of Parameters):

Not relevant in this example. Uniqueness is defined modulo equivalence. Two sets of parameters, A and B, are called EQUIVALENT, if, under all inputs the respective A-mode is indistinguishable from the respective B-mode (with respect to a pre-defined error margin).

FIG. 24:

Skipped, because in this example only local calibration is necessary.

FIG. 25:

D 2100, D 1200, D 1300, D 1050, D 1051:

As before.

D 1010 (Factual BAI-Dynamics):

Using data from ongoing measurements of brain activity, Pos is calculated on an ongoing basis (ONGOING=within suitable time-periods).

$$Pos \text{:} = {\sum }_{i = 13}^{30} {f}_{i}^{2} / {\sum }_{i = 8}^{12} {f}_{i}^{2}$$

according to S 500.

S 2210, S 2120:

As before

D 4100, D 4200:

As before

D 4300:

Can not be realized with just one transmitter. Will be explained in example 3.

S 3100 (Calculation of one or more transmitter signals):

The normally infinite plurality of possible transmitter signals is reduced analogously to the calculation of test signals. In the easiest case, transmitter signals will be used, which have already been used as test signals. Transmitter signals will be tested for feasibility (D 4200, D 4100, D 4300), and sorted with respect to the pre-defined utility function (e.g. frequency as low as possible, field strength as low as possible, etc.). The best possible transmitter signal will be selected automatically. Example: the abovementioned feedback signal, transmitted by T₁ with 0.4 Tesla will result in achieving the behavioral target, because Pos will be more than doubled (compare FIG. 12(i) with FIG. 17(iv)).

S 3400 (Transmission):

Transmitting the signal(s) chosen in accordance with S 3100 (here: by means of transmitter T₁).

D 4040 (Prediction of BAI Dynamics):

The model modes with exogenous inputs determine the related brain activity over time, and generate a time series. From the future parts of this time series, theoretical BAI dynamics are calculated as before, which serves as predicted BAI dynamics. In the example given only one model mode is relevant. The prediction of BAI dynamics is therefore calculated by dividing the BAI shown in FIG. 13(i) by a attenuation factor 10/2.2.

S 3500 (Comparison):

On one hand, factual BAI dynamics from S 2120 are subtracted from the predicted BAI-dynamics D 4040. For each frequency between 1 Hz and 50 Hz the absolute value of this difference is calculated, the maximum thereof is the result of the comparison. On the other hand, whether the behavioral target (the target BAI dynamics) has been achieved, will be calculated analogously by comparing factual- and target-BAI-dynamics.

E 300 (Difference Acceptable?):

If the assumptions and the results of calibration are correct, it is to be expected that predicted BAI and factual BAI only differ marginally (difference below a predefined threshold), in this case one can continue to transmit (S 3400). If they differ substantially, the equations of the brain model need to be solved numerically with other endogenous inputs (S 3600), in order to test whether jumps of the piecewise continuous stationary endogenous input might explain the difference.

If the behavioral target is not achieved within the time period calculated, there will be a transition to S 3800, otherwise the message “behavioral target achieved” will be communicated.

S 2000 (Calibration):

As explained before.

Concluding remark for this example: the brain activity target will be achieved in a reliable way by calibration and subsequent controlled modification, and therefore, in case of applicability of the reduced Davidson-model, also the behavioral target.

### EXAMPLE 2

### (Steps Will Be Explained Only in Extracts):

FIG. 20:

D 2000 (Behavioral Target):

Amplification of positive and reduction of negative emotions for the duration of the application

D 3000 (Behavioral Model):

Davidson-model (see e.g. [11]): positive emotions correspond to a higher quotient of beta to alpha activity in the left-frontal cortex (beta=13-30 Hz, alpha=8-12 Hz), negative emotions correspond to a higher quotient of beta to alpha activity in the right-frontal cortex.

S 500 (Specify BAI, Specify Target BAI Dynamics):

In analogy to example 1. Derived BAI are

$$Pos \text{:} = {\sum }_{i = 13}^{30} {f}_{i , 1}^{2} / {\sum }_{i = 8}^{12} {f}_{i , 1}^{2}$$

for one left-frontal sensor S₁, and for

$$Neg \text{:} = {\sum }_{i = 13}^{30} {f}_{i , 2}^{2} / {\sum }_{i = 8}^{12} {f}_{i , 2}^{2}$$

a right-frontal sensor S₂. (fᵢⱼ coefficient of the i-Hertz mode of the sensor j). Pos shall be increased, e.g. Pos(t, with influence)>2*Pos(t, without influence). Neg shall be decreased, e.g. Neg(t, with influence)>0.5*Neg(t, without influence). These requirements determine the target BAI dynamics. Calculation as in example 1.

D 4000 (Minimal Device Requirements):

Surface-EEG with one electrode S₁ left-frontal, and a reference-electrode at the left ear, one electrode S₂ right-frontal, and a reference-electrode at the right ear (see [16]), two extracranial coils (transmitters T₁ and T₂), with e.g. T₁ directly adjacent to S₁, on a hypothetical line on the surface of the head, connecting S₁ and a central electrode Cz (s. [16]). T₂ analogously between S₂ and Cz, with the distance from S₁ to T₁ equal to the distance from S₂ to T₂. Everything else as in example 1.

D 2100 (Target BAI-Dynamics):

Pos(t, with influence)>2*Pos(t, without influence), and Neg(t, with influence)<0.5*Neg(t, without influence).

D 2200 (BAI Calculation Rules):

As in example 1, plus

$$Neg \text{:} = {\sum }_{i = 13}^{30} {f}_{i , 2}^{2} / {\sum }_{i = 8}^{12} {f}_{i , 2}^{2}$$

for the second, right-frontal sensor.

D 1000 (Generic Brain Activity Model):

Simplified Wilson-Cowan-model, as in example 1.

S 800 (Specification of Minimum Requirements for Calibration):

τ, a, b, c, d, ρₓ ρ_{y}, translation operators for four sensor-transmitter-pairs S₁-T₁, S₁-T₂, S₂-T₁, S₂-T₂. Only local calibration is necessary, because both frontal brain areas will be assumed to be independent (simplification). Their respective endogenous input will be assumed as constant.

D 1100 (Minimal Calibration Requirements):

Locally calibrate τ, a, b, c, d, ρₓ ρ_{y}, translation operators for four the sensor-transmitter-pairs S₁-T₁, S₁-T₂, S₂-T₁, S₂-T₂.

E 100 (Calibration Necessary?):

Local calibration is necessary.

D 4100 (Device Restrictions):

In analogy to example 1, on the output side e.g. two coils, each with a maximal magnetic field of 0.5 Tesla.

D 4200 (Health Limits for Exogenous Electromagnetic Fields):

As in example 1.

S 2000 (Calibration):

Will be explained later.

D 1200 (Specific Brain Activity Model):

As in example 1.

D 1300 (Quantification of the Influence of Exogenous EM Fields on Brain Activity):

As in example 1.

S 3000 (Control or Regulation of Brain Activity by Means of Targeted Modification of Control Variables and Measurement/Calculation of BAI):

Will be explained in detail when walking through the respective flow diagram (FIG. 25).

FIG. 21:

D 1100 (Minimal Calibration Requirements):

As before.

D 1110 (Calibration Type):

Local.

D 1120 (Calibration Range):

Sensors S₁, S₂, transmitters T₁, T₂.

E 100 (Calibration?):

Yes.

S 2100 (Local Calibration):

In analogy to example 1, for the four sensor-transmitter-pairs S₁-T₁, S₁-T₂, S₂-T₁, S₂-T₂. When performing local calibration four times, it is a substantial simplification that parameters and endogenous input of modes, which are detectable by S₁, can already be detected when locally calibrating the pair S₁-T₁. Therefore they can be considered as given when calibrating S₁-T₂ (amongst other processes: when decomposing). Analogous for modes within the detection range of S₂. It is recommended to start local calibration of a sensor with the transmitter, which has the minimal spatial distance to this sensor.

A result of a local calibration may be, e.g.:

For sensor S₁:

Mode I from example 1 with influence of T₁ calibrated as 40* [field strength at location of T₁, in Tesla]*xi₁(t), and influence of T₂=0. (xi₁ (t) is the time-delayed feedback of the signal measured by S₁, in reconstructed form).

For sensor S₂:

Mode II from example 1 with influence of T₂ calibrated as 40* [field strength at location of T₂, in Tesla]*xi₂(t), and influence of T₂=0. (xi₂ (t) is the time-delayed feedback of the signal measured by S₂, in reconstructed form).

S 2300 (Identification of Identical Brain Activities):

Above modes are not identical.

E 120 (Translocal Calibration?):

No.

D 1200 (Specific Brain Activity Model):

As before.

D 1300 (Quantification of the Influence of Exogenous EM Fields on Brain Activity):

As before.

FIGS. 22-23:

As before, respectively as just disclosed under S 2100.

FIG. 24:

Skipped, because in this example only local calibration is needed.

FIG. 25:

In analogy to example 1, with the exception of S 3100, which shall be explained in the following: e.g. the combined signal of T₁, xi₁(t), with 0.4 Tesla, and of T₂, xi₂(t), with 0.4 Tesla induce for mode I an increase of Pos to more than 200% of its original value (see example 1), for mode II a decrease of Neg to less than 50% of the original value (compare FIG. 12(ii) with FIG. 18(iv)). The brain activity target is thereby achieved, likewise, in case of validity of the Davidson-model, the behavioral target.

### EXAMPLE 3

Example 3 shall not be presented as comprehensively as the previous examples (e.g. all modes, local calibration of all sensor-transmitter-pairs, etc), but shall mainly highlight essential differences with respect to the previous examples.

FIG. 20:

D 2000 (Behavioral Target):

Prolongation of focused attention on mental activities for the duration of the application.

D 3000 (Behavioral Model):

Based on e.g. [20] and [21]), FmΘ(frontal midline theta), i.e. 6-7 Hz activity in the vicinity of the Fz-electrode (see [16]), corresponds to focused attention on mental activities. This fact shall in the following be called Ishihara-Yoshii-model.

Neurons near the cranium, within the detection range of the Fz-sensor, are directly connected with some other areas, in particular subcortical driving was detected. The power spectrum is calculated as before. Instead of Pos and Neg there is now for each sensor

$$Att \text{:} = {\sum }_{i = 6}^{7} {f}_{i}^{2} / {\sum }_{i = 8}^{max} {f}_{i}^{2} ( max = 50 ) .$$

D 4000 (Minimal Device Requirements):

In order to determine possible spreading phenomena it makes sense to augment the Fz electrode by at least one neighboring electrode, the detection range of which has a direct physiological connection with the detection range of the Fz electrode (e.g. the Cz electrode), i.e. surface EEG with two measurement electrodes and two reference electrodes (as shown in [21]). Two coils (transmitters) are localized in the immediate vicinity of the respective electrode: T₁ anterior with respect to Fz, T₂ posterior with respect to Cz. Everything else as in example 1.

D 2100 (Target BAI-Dynamics):

E.g. Att₁(with influence)=Att₁(without influence, with full driving), Att₂(with influence)<=Att₂(without influence), Here “1” denotes “Fz and reference electrode”, “2” denotes “Cz and reference electrode”, “=” denotes “difference between right-hand-side and left-hand-side under 20%”.

D 2200 (BAI Calculation Rules):

Power spectrum as in example 1, plus

$$Att \text{:} = {\sum }_{i = 6}^{7} {f}_{i}^{2} / {\sum }_{i = 8}^{max} {f}_{i}^{2}$$

for each of both sensors.

D 1000 (Generic Brain Activity Model):

Simplified Wilson-Cowan-model, as in example 1, but now with focus on translocal effects, and without the assumption, that endogenous input should be constant. Endogenous input into the excitatory subpopulation of the j-th neural oscillator can, in the easiest case of translocal calibration, be split into

$${ρ}_{{x}_{j} , endo} ( t ) \text{:} = {\sum }_{l} {h}_{jl} \cdot {x}_{l} ( t - {Δ}_{jl} ) ,$$

where “hⱼₗ” is the strength of the influence of the l-th on the j-th neural oscillator, and the respective A is the time delay, until this influence becomes effective.

S 800 (Specification of Minimum Requirements for Calibration):

As in example 2, plus functional matrix. Local and translocal calibration are necessary.

D 1100 (Minimal Calibration Requirements):

As in example 2, plus functional matrix.

E 100 (Calibration Necessary?):

Yes.

D 4100 (Device Restrictions):

As in example 2.

D 4200 (Health Limits for Exogenous Electromagnetic Fields):

As in example 2.

S 2000 (Calibration):

Will be explained when walking through the flow diagrams of FIGS. 21-24. Stationarity of endogenous input during calibration, not during the whole application, is assumed.

D 1200 (Specific Brain Activity Model):

As in example 1.

D 1300 (Quantification of the Influence of Exogenous EM Fields on Brain Activity):

As in example 1.

S 3000 (Control or Regulation of Brain Activity by Virtue of Targeted Modification of Control Variables and Measurement/Calculation of BAI):

Will be explained in detail when walking through the respective flow diagram (FIG. 25).

FIG. 21:

D 1100 (Minimal Calibration Requirements):

As before.

D 1110 (Calibration Type):

Translocal (includes local).

D 1120 (Calibration Scope):

As in example 2.

S 2100 (Local Calibration):

Will be explained in detail when walking through the flow diagrams of FIGS. 22-23.

S 2300 (Identification of Identical Brain Activities):

In analogy to example 2.

E 120 (Translocal Calibration?):

Yes.

S 2400 (Translocal Calibration):

Will be explained in detail when considering FIG. 24.

D 1200, D 1300:

As in example 2, plus functional matrix.

FIG. 22:

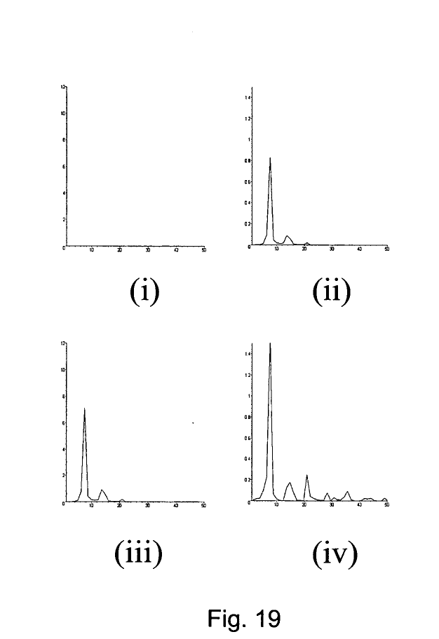

In analogy to example 1. Now a BAI model mode within the detection range of S₁ shall be of interest, with underlying parameters τ=10, a=16, b=20, c=8, d=3, with endogenous input ρₓ=1.5+m*sin(2*π*t*7/1000) and ρ_{y}=1.5+m*sin (2*π*t*7/1000), shown for m=0 in FIG. 19(i), for m=2 in FIG. 19(ii), and for m=4 in FIG. 19(iii) (In this example it is assumed that subcortical driving with 7 Hz has the same impact on the excitatory subensemble as it has on the inhibitory subensemble). This mode shall be denoted as mode III. Test signals close to the 7 Hz sinusoidal driving are possibly suitable, e.g. a 7 Hz sine signal, which every 10 milliseconds, is switched off for 5 milliseconds, in order to permit measurements. Such a test signal, transmitted with 0.5 Tesla by transmitter T₁, shall have an influence on mode III with a multiplier of 16, by transmitter T₂ with a multiplier of zero.

Here local calibration includes phase-shifting of test signals. The BAI minimum of the driven signal provides the phase shift theta necessary to switch off the mode. E.g. said sine signal will be transmitted with a phase shift of theta at 0.5 Tesla by transmitter T₁, the result of this is shown in FIG. 19(i).

Only S₂ mode is e.g. the S₁ mode from example 1. Both said T₁-signals shall not have an impact on the S₂ mode. A 0.1 Tesla sine signal from T₂ shall not have an impact on the theta-mode.

FIG. 23:

In analogy to example 2.

FIG. 24:

D 1300 (Translation Operators):

If i and k are different, these translation operators are equal to zero for both signals shown for FIG. 22.

S 2410 (BAI Prediction):

As before

For the S₁-mode: S 2420 (Modify):

Example: Transmission of a 0.5 Tesla 7 Hz sine signal by T₁, phase-shifted by theta, will switch off the S₁-mode.

For the S₁-mode: S 2430 (Determine S₂-gap):

The BAI prediction for S₂ is “unchanged continuation”, as the signals have no direct impact on the S₂ mode. This prediction shall coincide with the factual BAI for S₂.

For the S₂-mode: S2420 (Modify):

Example: shift the frequency of the S₂ mode to a basic frequency, which is incompatible with 7 Hz, e.g. by time-delayed feedback of the signal measured by S₂, with reverse-polarity, transmitted by T₂.

For the S₂-mode: S 2430 (Determine S-gap):

The BAI prediction for S₁ is “unchanged continuation”, as the T₂ signals have no direct impact on the S₁ mode. This prediction shall coincide with the factual BAI for S₂. This results e.g. in a change of the signal component shown in FIGS. 19(iii) towards 19(ii).

S 2440 (Calculating Functional Matrices, which Explain the Gap):

No gap in case of changes of S₁, therefore the cell (1,2) equals zero (mode I=S₁ mode shown, mode 2=S₂ mode shown). Non-unique gap for cell (2,1).

E 400 (Unique?):

Clarify whether one or more functional matrices result from S 2440.

If, for the respective application, one is not interested in general correspondences between brain activities, but in specific ones, e.g. between brain activities i and j, it is sufficient to unambiguously fill the cells (i,j) and (j,i) of the functional matrix.

In the example given, it is only of interest, whether BAI-preserving modifications of mode I spread, i.e. change mode II. For this purpose, consideration of cell (1,2) is sufficient—which has the value zero.

S 2221 (Change Signal):

E.g. generation of frequency shifts, amplitude changes, changes in stationarity, addition of noise, change of Lyapunov exponents, and many more (not needed in this example).

D2410 (Functional Matrix):

The result comprises of one functional matrix each, which here e.g. has the following shape:

$$( \begin{matrix}- & 0 \\ undetermined & -\end{matrix} ) .$$

FIG. 25:

In analogy to example 2. In addition to this, by means of translocal calibration, it has been determined, that a spreading restriction (D 4300) of the type: “Changes caused by T₁ shall not affect other brain activities than those captured by S₁” is fulfilled. In the example given, FmΘ, in case of driving decreased to zero (see FIG. 19i) is stabilized near its natural spectra (“full driving”, s. FIG. 19,(ii) and (iii)) by means of the said intermittent sine signal by T₁, without perturbing the brain activity detected by S₂. The result (measured by S₁) is shown in FIG. 19(iv). The brain activity target of restoring Att₁, without perturbing Att₂, is thereby achieved, the same applies to the behavioral target in case of applicability of the Ishihara-Yoshii-model.

Further explanations and remarks:

Ad D 1000: A brain activity model is, in the easiest case, a set of difference- and/or differential equations e.g. of the type
\dot{x}(t)=ƒ(x,t,localParameters,endogenousInput,exogenoutInput)
for a brain activity. x brain activity index, t time. “Exogenous input” refers to artificially (by virtue of extracranially generated electromagnetic fields) generated values of input variables in the equations of the brain activity model (as opposed to “endogenous input”, which normally is transmitted via sensory or nervous channels, e.g. by stimulation with light, by thalamic pacemaking, by listening to a symphony, and many more). Exogenous input=Ü (transmitter signal), Ü translation operator. The function f depends on the brain activity model used, and determines the time derivative of x by virtue of x, t, and non-observable endogenous and exogenous input, with a non-observable set of parameters. General brain activity models use paths instead of point-like x, and/or stochastic differential equations, which include noise.

Different brain activity models lead to different calibration results, with possibly different modifications. In case of suitable brain models, the brain activity target will be achieved in a reliable way, in case of a suitable behavioral model, the behavioral target will be achieved in a reliable way. Brain activity models and behavioral models can also be represented in integrated form.

Ad S 2000:

The nonlinearity of the system allows for determination of non-observables, including the translation operator, by suitable variation of a transmitter signal (“test signal”) with field strengths as small as possible.

The dynamic calibration presented here differs from existing mathematical methods for parameter estimation (these, in general, make distribution assumptions for parameters, which are regarded as random variables, to then observe evolution along trajectories, and by assumption of convergence of parameter values calculated from these observations towards “true” parameters, estimate the later, see, e.g. [7]), and from adaptive control/parameter estimation known from engineering (these are, in general, useful only for linear systems, where elementary control functions are entered into the system to minimize the difference between ideal target value and observed value, see e.g. [8]).

Dynamic calibration differs from “passive observation” (conventional non-invasive observation) used in the above mathematical approaches by using “active measurement” (transmission of signals in to the system and measurement of the system in interaction with the signals). The result of active measurement (e.g. stochastic resonance) is in many cases of nonlinear deterministic and/or nonlinear stochastic systems (like the human brain) very different from responses of linear systems to the usual Dirac- or Heaviside-inputs, or the like.

There are major differences between known adaptive control/parameter estimation and dynamic calibration:
1st Dynamic calibration is normally preceding control (and a process separate from control),
2nd in dynamic calibration the emphasis is on the test functions used, the plurality of which (and thereby their ability to discriminate between different sets of non-observables) matches the complexity of the system considered (as opposed to simple test functions like Heaviside-functions, where often the system response needs to be observed for a long time, which is unacceptable in-vivo),

3rd the type of approximation of the underlying parameter set is completely different (for dynamic calibration successive elimination within subsets of a discretized parameter manifold, as opposed to target/actual-comparison of control parameters).

In addition to determining non-observables, dynamic calibration will determine the otherwise unknown translation operators, which are essential in heterogeneous and/or complex unknown systems to translate characteristics of artificially generated fields (e.g. field strength, and/or frequency pattern of extracranial magnetic fields) into exogenous input in the system's equations.

Ad D 2410:

For m brain activities observed, a functional matrix is a m*m-matrix, with a cell (ij) equal to zero, if no functional connection from activity j to activity i could be determined.

Functional connections between brain activities require physiological connections of the underlying brain areas, but the existence of the later does not necessarily result in a functional coupling.

Functional matrices are admissible, if (after discretization of parameters) they result in solutions compatible with observations for the totality of observed brain activities. Here, in the equations for each brain activity, endogenous input was replaced by, e.g., residual input+sum of all upstream activities times their respective influence strengths times zⱼ (t-delay) (“A upstream with respect to B” if altering A changes B, equivalent to: “B downstream with respect to A”).

A preferred embodiment of the method for local modification includes the following elements (assuming that a cluster of adjacent Wilson-Cowan oscillators can be modeled by a pair of Wilson-Cowan equations):
a) data analysis by means of Fourier analysis, alternatively in combination with wavelet analysis (suitable linear combinations of x(t) and y(t) as basis, fundamentals see [13]), phase space embeddings, and statistical methods, in order to (with respect to said methods of analysis) determine the probability of the measured signal belonging to an equivalence class of solutions of the Wilson-Cowan equations. Preferred test signals are linear combinations of time-delayed feedback signals (using the measured signal) and/or solutions of the Wilson-Cowan equations, μ*(X(t−τ)−y(t−τ)).
b) local calibration,
c) modification, as described,
d) ongoing seizure-warning plus automatic preventive control, like in e.g. [9].

Analogous to this is the preferred embodiment for translocal phenomena, as described above, plus translocal calibration.

If no calibration is needed, and if the limit cycle oscillators are weakly coupled, and if the assumption is valid, that for small periods of time clusters of weakly coupled limit cycle oscillators can be replaced by clusters of phase oscillators, instead of limit cycle oscillators (solutions of the Wilson Cowan equations) coupled phase oscillators (e.g. [23]) can be used. Interventions are based on phase resetting and subsequent external slaving plus ongoing measurement of a signal, such that the cluster will converge towards the target. This embodiment is applicable preferably for clearly circumscribed brain areas, such that the reset would not generate unwanted disturbances.

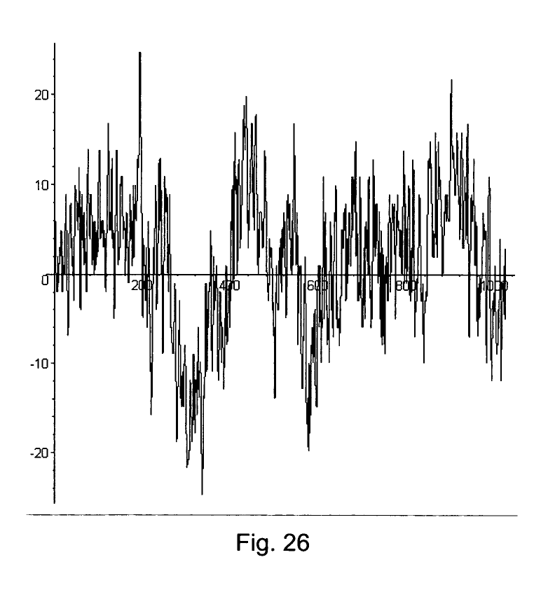

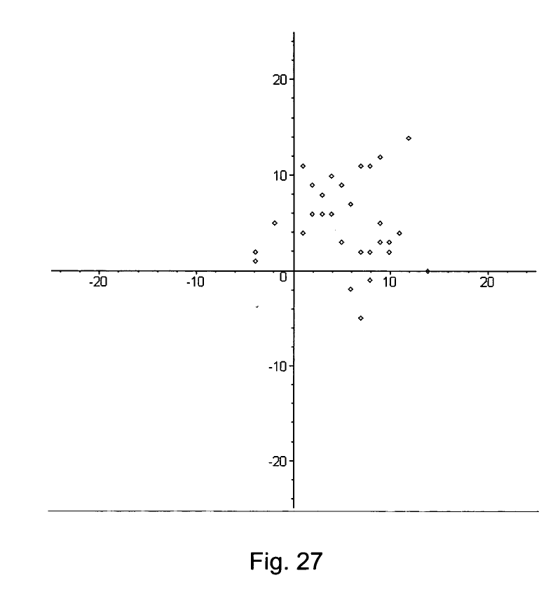

For one input channel phase space embedding shall be illustrated: FIG. 26 shows the EEG data of one channel, with time units (1/128 of a second) on the x-axis, differences in potential (between measurement electrode and reference electrode at the respective point in time) on the y-axis. In the easiest case one or more orbits are determined according to [29]. In general the embedding is performed via delay coordinates (see [27]). The delay time can be determined as the first zero-crossing of the autocorrelation function (see [29]): embedding of a data window for t=130 and τ=86 (s. FIG. 27). Here the x-axis shows a window, ending with t, of length 32α·x(t−86)+β·y(t−86), and the y-axis shows α·x (t)+β·y (t). In FIG. 27, α is given as 1, β is given as −1. As multiple occupation of the same point is not shown, the number of points in FIG. 27 is smaller than 32. By virtue of known methods (Bayes, Minimax, and many others), and after rescaling the degree of coincidence of the empirical phase space occupation with every (on the finite parameter grid) solution of the Wilson-Cowan equations for each (on a discretized interval) mixing angle is determined.

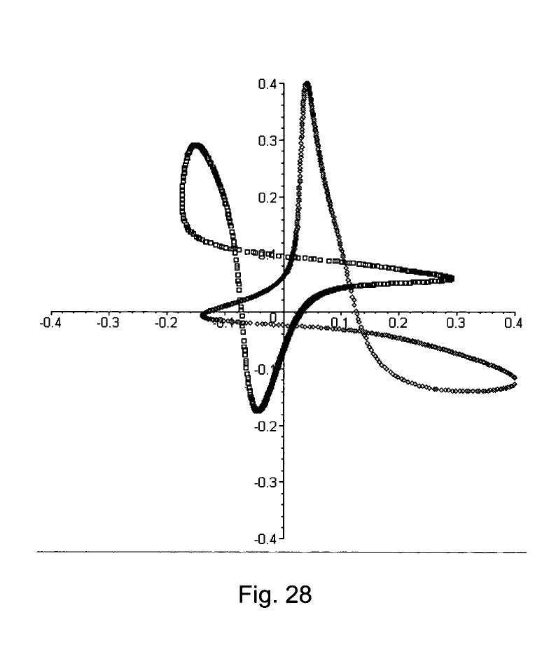

FIG. 28 (with the same axes as in FIG. 27) and the same mixing angles shows the solutions for τ=10, a=22, b=20, c=14, d=3, ρₓ=1.5, for several ρ_{y}: rhombi for ρ_{y}−1.5, squares for y=0.

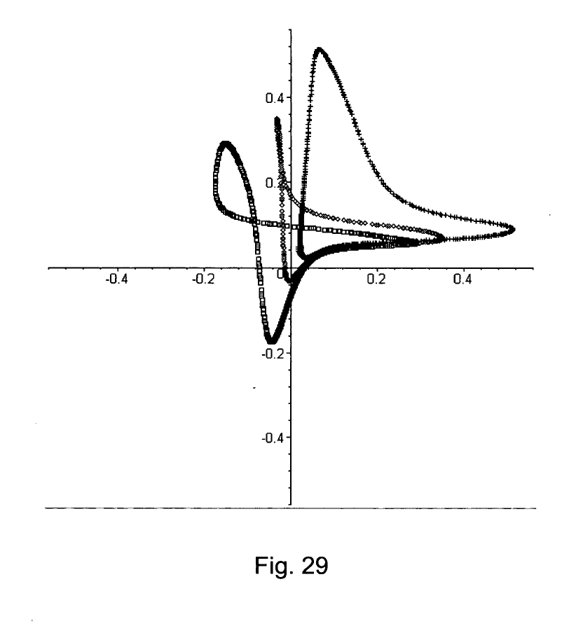

FIG. 29 demonstrates the importance of the mixing angles: here one obtains, with the same notation as before, the solutions for τ=10, a=22, b=20, c=14, d=3, ρₓ=1.5, ρ_{y}=0, for \acute{α}=1 and β=−1 (rhombi), as well as β=−0.7 (squares), and β=−0.3 (crosses).

In an preferred embodiment of the method, instead of the Wilson-Cowan equations, stochastic Wilson-Cowan equations will be used, i.e. ρₓ=ρ_{x,det}+noise, ρ_{y}=ρ_{y,det}+noise. “det” denotes the deterministic part of the input, “noise” stochastic input. The same method as before will be applied. In addition to this, the noise level will be determined. Here it also makes sense to use noisy test signals and to measure signal-to-noise ratios.

In another preferred embodiment of the method in the Wilson-Cowan equations different time constants τ will be used for x and for y, and/or different multipliers μ. The calibration of these will be carried out as it has already been described for the other parameters.

In another preferred embodiment of the method in the Wilson-Cowan equations into each Wilson-Cowan-neuron of a cluster the other neurons of the cluster will be coupled into via mean-field-coupling, i.e. for all i=1.N

$$τ {\dot{x}}_{i} = - {x}_{i} + S ( {ax}_{i} + {by}_{i} {ρ}_{x , i} + ɛ {\sum }_{j = 1 , j \neq i}^{N} {x}_{j} + δ {\sum }_{j = 1 , j \neq i}^{N} {y}_{j} ) ,$$

as well as

$$τ {\dot{y}}_{i} = - {y}_{i} + S ( {cx}_{i} + {dy}_{i} {ρ}_{y , i} + η {\sum }_{j = 1 , j \neq i}^{N} {x}_{j} + 𝓋 {\sum }_{j = 1 , j \neq i}^{N} {y}_{j} ) ,$$

with

ε,δ,η,ν new non-observables, which need to be determined. Analogous to this, this embodiment can be implemented for stochastic Wilson-Cowan-neurons, with or without time-delayed input.

In another preferred embodiment of the method the shape of the sigmoid function of the neurons of a cluster will be calibrated by using noisy test functions, because dependent on the sigmoid function the cluster amplifies or dampens signal respectively noise with different strength (for simple networks see e.g. [28]).

In another preferred embodiment of the method a test function and this test function with reversed polarity are used one after the other, i.e. first transmitting a signal xi(t), then −xi(t).

In another preferred embodiment the calibration is stopped after n>0 test signals (“fast calibration”), and one set of parameters and endogenous and exogenous inputs compatible with the measured data is set as preliminary calibration result. Additional measured data, which arrive as a result of performing modification (S3000), will be used for adaptation of the calibration on an ongoing basis.

In another preferred embodiment the measurement-assumptions and -invariants (D 1049, D 1050) will be validated.

In a preferred embodiment the EM method is combined with sensory input (acoustic, optical, etc.). Example: instruction to the user in example 3, to start a mental task (see e.g. [20]) at the beginning of the calibration, whereby the task of finding suitable FmΘ-modes is greatly simplified. Another example is acoustic pacemaking for translocal calibration of auditory brain processes.

In a preferred embodiment the effect of the method will be stabilized beyond the duration of the individual application sessions, by using suitable repetition rates, which need to be determined for each user by means of external validation. This consequence of suitable repetition rates is known e.g. for TMS applications (transcranial magnetic stimulation with magnetic fields of 1-2 Tesla), see, e.g. [3].

In an preferred embodiment calibration is combined with sensor optimization: activating previously inactive sensors and/or position changes of sensors and/or changes in the orientations of sensors such that identified and/or non-identified brain activities will be measurable particularly well by the respective (if necessary newly oriented) sensor. For sensor optimization conventional optimization methods are suitable.

In a preferred embodiment calibration is combined with sensor optimization by combining several existing sensors to virtual sensors, such that brain activities will be measurable particularly well by the respective virtual sensor. Example: S₁, S₂, S₃, S₄ combined via f(S₁, S₂, S₃, S₄)=0.3*S₁+0.5*S₂+0.02*S₃*S₃+0.5*sin(S₁+S₄) to Sᵥᵢᵣₜᵤₐₗ. A suitable f is calculated for each subset of sensors by conventional optimization methods.

In a preferred embodiment calibration is combined with transmitter optimization: activating previously inactive transmitters and/or position changes of transmitters and/or changes in the orientations of transmitters such that brain activities will become influenceable particularly well by the respective (if necessary newly oriented) transmitter. For transmitter optimization conventional optimization methods are suitable.

In a preferred embodiment calibration is combined with transmitter optimization by combining several existing transmitters to virtual transmitters, such that brain activities will be influenceable particularly well by the respective virtual transmitter. The optimum (the virtual transmitter) is calculated from the translation operators by using conventional optimization methods.

In a preferred embodiment of S 2130 the decomposition is carried out for n sensors in parallel (n integer between 1 and the total numbers of sensors used), therefore the time series to be decomposed is an n-tuple of 1-sensor-time-series.

In a preferred embodiment of S 2130 the decomposition is performed for an n-tuple of 1-sensor-time-series by using pursuit methods (e.g. matching pursuit, see [13]).

In another preferred embodiment of S 2130 the decomposition of the n-tuple of 1-sensor-time-series is performed with the Karhunen-Loeve-method (see e.g. [17], [18]) and/or independent component analysis (“ICA”, see e.g. [22]).

In another preferred embodiment of S 2130 the decomposition is performed after an embedding into a meta-phase-space, whereby stationary and non-stationary components are separated (see e.g. [15]).

In another preferred embodiment of S 2130 the decompositions are carried out in parallel, with several methods, whereby only modes which have been identified by a majority (under a pre-defined weighting) of these methods will be accepted for further processing (e.g. for relevance testing).

In a preferred embodiment of D 2000 altering or sustaining perception and/or the perceptive capacity and/or the perceptive capability will be set as behavioral target. Example for this is the modification of the ability to discriminate between specific stimuli or classes of stimuli.

In a preferred embodiment of D2000 altering or sustaining actions and/or the action capacity and/or the action capability will be set as behavioral target. Example is here improving reaction speed.

In a preferred embodiment of D2000 altering or sustaining activation and/or activation capacity will be set as behavioral target.

In another preferred embodiment of D2000 altering or sustaining motivation and/or motivation capacity and/or the motivation capability will be set as behavioral target.

In another preferred embodiment of D2000 altering or sustaining attention and/or attention capacity will be set as behavioral target.

In another preferred embodiment of D2000 altering or sustaining memory and/or memory contents and/or the retrieval of memory contents will be set as behavioral target.

In another preferred embodiment of D2000 altering or sustaining learning and/or the learning capacity and/or the learning capability will be set as behavioral target.

In another preferred embodiment of D2000 altering or sustaining consciousness will be set as behavioral target.

In another preferred embodiment of D2000 altering or sustaining emotions and/or the emotional capacity and/or emotional capability will be set as behavioral target.

In another preferred embodiment of D2000 altering or sustaining appetencies and/or aversions will be set as behavioral target.

In another preferred embodiment of D2000 altering or sustaining cognition and/or the cognitive capacity and/or the cognitive capability will be set as behavioral target. (“Cognition” is defined here as “carrying out mental processes”).

In another preferred embodiment of D2000 altering or sustaining behavioral sequences will be set as behavioral target.

In another preferred embodiment of D2000 altering or sustaining behavioral correlations will be set as behavioral target.

In another preferred embodiment of D2000 several compatible behavioral targets will be combined.

In another preferred embodiment of D 2000 several not necessarily compatible behavioral targets will be combined hierarchically, i.e. a priority list of behavioral target will be generated. In case of mutually inconsistent procedural steps the step which is associated with a behavioral target of lower priority will be carried out later or not at all.

In another preferred embodiment of D 2000 the list of behavioral targets is augmented with the top-priority target of non-occurrence of seizures. This includes an application of [9] to users without a respective medical record.

In another preferred embodiment of D 3000 the behavioral model is taken from the very substantial body of literature (e.g. for the examples presented from [11], [20], [21], there are dozens of publications with the same/similar subject).

In another preferred embodiment of D 3000 several behavioral models will be used in parallel, whereby only these controlled or regulated, respectively, steps (S 3000) which are compatible with the majority (under a pre-defined weighting) of these behavioral models, will be performed.

In a preferred embodiment there is an external validation (normally using psychological tests) of the alteration or sustenance of behavior for the individual user during or after the EM application. Thereby it is tested, to which extent the behavioral model used (which in general has been validated on a statistical basis) is valid for the respective user.

In another preferred embodiment of D 1000 several generic brain activity models will be used in parallel, whereby only these controlled or regulated, respectively, steps (S 3000) which are compatible with the majority (under a pre-defined weighting) of these models, will be performed.

In another preferred embodiment of the method in the steps concerning single sensors and/or single transmitters, these will be replaced by set of sensors and/or transmitters. The purpose of this is determination and controlled modification of non-local spatio-temporal modes, which occur e.g. in Jirsa-Haken-models (see e.g. [18]).

In another preferred embodiment of D 4000 the minimal device requirements will be exceeded, in order to obtain additional information, which is potentially useful for further applications with possibly changed behavioral targets (e.g. always using 10/20 surface EEG, see e.g. [16]). The advantage of this embodiment is a possible standardization of parts of the device as well as usability of commercially available hardware.

In another preferred embodiment of D 4000 the minimal device requirements will be exceeded, in order to obtain additional possibilities for control or regulation (e.g. for each non-reference electrode of 10/20 surface EEG to have one coil immediately frontal to the electrode). The advantage of this embodiment is a possible standardization of parts of the device.

In another preferred embodiment of S 3000 the exogenous EM fields giving rise to control variables are augmented by sensory inputs and/or biochemical active compounds, whereby the procedural steps described do not change in principle.

In another preferred embodiment of the method test- and transmitter-signals with stochastic components will be used (especially together with brain activity models with explicit stochastic components, as e.g. [25], an augmentation of [18], with the target of utilizing stochastic resonance and stochastic coherence, see e.g. [26] for the FitzHugh-Nagumo-model).

In another preferred embodiment of the method test- and transmitter-signals with chaotic components will be used (especially together with brain activity models with explicit chaotic components, as e.g. enhancements of [1] with local strong coupling between oscillators).

In another preferred embodiment of the method inactive modes will be activated directly by transmitter signals, i.e. by the direct effect of the signals on the mode.

In another preferred embodiment of the method modes will be influenced indirectly by transmitter signals, i.e. by the direct effect on modes which induce (see functional matrix) effects on the mode to be influenced indirectly.

In another preferred embodiment of the method bifurcation points will be controlled, i.e. brain activity will be changed qualitatively (like in example 3, where the neural oscillator, which was inactive without driving, was switched on).

In another preferred embodiment of the method inactive modes will be activated.

In another preferred embodiment amplitudes will be amplified by activating inactive modes and/or synchronizing modes, analogously reduced by switching modes off and/or desynchronizing modes.

In another preferred embodiment amplitude amplification is used-against age-correlated flattening (for EEG, see e.g. [12]).

In another preferred embodiment of the method non-stationary signals are decomposed into a stationary and a non-stationary part (see e.g. [15]), whereby the test- and transmitter-signals are to be modulated with the non-stationary part.

In another preferred embodiment of the method users are identified on the basis of calibration data, which are stable over time. Even conventional spectrally decomposed EEG-data for an individual have components, which are stable over time (experimentally validated for up to 5 years, e.g. [10]).

Relevant Publications:

[1] “Excitatory and inhibitory interactions in localized populations of model neurons”, Wilson H R, Cowan J D, Biophysical Journal 1972; 12: 1-22

[2] WO00/29970 A1

[3] “Therapeutic application of repetitive transcranial magnetic stimulation : a review”, Wassermann E M, Lisanby S H, Clin Neurophysiol 2001; 112: 1367-1377

[4] WO98/18384 A1

[5] “Anticipation of epileptic seizures from standard EEG recordings”, Le Van Quyen M, Martinerie J, Navarro V, Boon P, D'Have M, Adam C, Renault B, Varela F, Baulac M, The Lancet 2001 Jan 20 ; 357: 183-8

[6] WO01/21067 A1

[7] “Numerical Solution of Stochastic Differential Equations”, Kloeden P E, Platen E, Springer, Berlin 1992, chapter 6.4

[8] “Regelungstechnik”, Follinger O, Hüithig Buch Verlag, 8th rev. ed., Heidelberg 1994, chapter 1.6

[9] DE 102 15 115.6

[10] “Intraindividual Specificity and Stability of Human EEG: Comparing a Linear vs a Nonlinear Approach”, Dünki R M, Schmid G B, Stassen H H, Method Inform Med 2000; 39: 78-82

[11] “An EEG Biofeedback Protocol for Affective Disorders”, Rosenfeld J P, Clin Electroencephalography 2000; 31 (1): 7-12

[12] “Leitfaden für die EEG-Praxis”, Ebe M, Homma I, Gustav Fischer, Stuttgart 1992, chapter 6.2. 2

[13] “A Wavelet Tour of Signal Processing”, Mallat S, Academic Press, San Diego 1999,

[14] “Applied Bioelectricity”, Reilly J P, Springer, New York 1998, chapter 11

[15] “Stationarity and nonstationarity in time series analysis”, Manuca R, Savit R, Physica D 1996; 99: 134-161

[16] “Leitfaden fir die EEG-Praxis”, Ebe M, Homma I, Gustav Fischer, Stuttgart 1992, chapter 3.6.1, and 3.6.2

[17] “Nonlinear Spatio-Temporal dynamics and Chaos in Semiconductors”, Schöll E, Cambridge University Press, Cambridge 2001, Kapitel 7.3. 4.

[18] “A derivation of a macroscopic field theory of the brain from the quasi-microscopic neural dynamics”, Jirsa V K, Haken H, Physica D1997; 99: 503-526

[19] “Approaches to verbal, visual and musical creativity by EEG coherence analysis”, Petsche H, Int J Psychophysiol 1996; 24: 145-159

[20] “Frontal midline theta rhythm and mental activity”, Inanaga K, Psychiatry and Clin Neurosciences 1998; 52: 555-566

[21] “Frontal midline theta rhythms reflect alternative activation of prefrontal cortex and anterior cingulate cortex in humans”, Asada H, Fukuda Y, Tsunoda S, Yamaguchi M, Tonoike M, Neuroscience Letters 1999; 274: 29-32

[22] “Imaging Brain Dynamics Using Independent Component Analysis”, Jung T P, Makeig S, McKeown, Bell A J, Lee T W, Sejnowski T J, Proceedings of the IEEE July 2001; 89 (7): 1107-1122

[23] “Desynchronizing double-pulse phase resetting and application to deep brain stimulation”, Tass P A, Biol Cybern 85, 2001: 343-354

[24] “Mathematical aspects of Hodgkin-Huxley neural theory”, Cronin J, Cambridge University Press, Cambridge 1987, chapters 2 and 3

[25] “Impacts of noise on a field theoretical model of the human brain”, Frank T D, Daffertshofer A, Beek P J, Haken H, Physica D 1999; 127: 233-249

[26] “Coherence and stochastic resonance in a two-state system”, Lindner B, Schimansky-Geier L, Physical Review E June 2000; 61 (6): 6103-6110

[27] “Analysis of Observed Chaotic Data”, Abarbanel HDI, Springer, New York 1996, Kapitel 3.3

[28] “Introduction to Neural Dynamics and Signal Transmission Delay”, Wu J, DeGruyter, Berlin 2001, chapter 3.4

[29] “Periodic Orbits: A New Language for Neuronal Dynamics”, So P, Francis J T, Netoff T I, Gluckman B J, Schiff S J, Biophysical Journal 74, June 1998: 2776-2785

While this invention has been particularly shown and described with references to preferred embodiments thereof, it will be understood by those skilled in the art that various changes in form and details may be made therein without departing from the scope of the invention encompassed by the appended claims.

1. A method for the electromagnetic modification of brain activity in order to achieve a behavioral target, using a behavioral model describing the correspondence between behavior and the dynamics of brain activity indexes, and using a brain activity model, which quantitatively describes brain activity, and from which brain activity indexes and their dynamics can be derived, the method comprising:
determining, by means of the behavioral model, which target dynamics of specific brain activity indexes correspond to achieving the behavioral target,
generating exogenous electric and/or magnetic fields, and measuring electromagnetic brain activity and calculating brain activity indexes, in order to achieve the target dynamics as part of an open or closed control loop, which includes brain model calculations.

2. A method according to claim 1, wherein a generic brain activity model is used, which, by determining non-observables for a specific user, is calibrated to a specific brain activity model, and wherein the influence of exogenous electric and/or magnetic fields on brain activity is determined for this user.

3. A method according to claim 2, wherein non-observables are calibrated for a specific user.

4. A method according to claim 2, wherein the calculation of the influence of an exogenous electric and/or magnetic field on the brain activity of a specific user is coupled to the determination of non-observables.

5. A method according to one of the claim 2, wherein calibration first includes a decomposition of factual dynamics of brain activity indexes into BAI-modes, such that these BAI-modes correspond to dynamics of brain activity indexes calculated from the brain activity model.

6. A method according claim 5, wherein the decomposition of an n-element time-series into spatio-temporal modes is carried out.

7. A method according to claim 5, wherein the decomposition is carried out with a pursuit method.

8. A method according to claim 5, wherein the decomposition is carried out with respect to the transformed output functions of the brain model, whereby each transformation represents the alteration of the signal between the neurons participating in the brain activity and the respective sensor.

9. A method according to claim 5, wherein the decomposition is performed after embedding into a meta-phase-space, whereby stationary and non-stationary components become separable.

10. A method according to claim 5, wherein the decomposition is performed with different methods, whereby the sets of modes will be weighted depending on the respective method, and only these modes will be employed further, the total weight of which is above a specific threshold.

11. A method according to claim 5, wherein, based on these modes, sets of parameters and endogenous inputs and/or exogenous inputs are determined, which may underlie the respective mode according to the brain activity model.

12. A method according to claim 2, wherein for several detection ranges local modes are calibrated, the interactions of which will be determined by successive modification of individual local modes.

13. A method according to claim 12, wherein amplitude amplification of age-correlated activity reduction is carried out. -

14. A method according to claim 2, wherein calibration requirements for individual sensors are replaced by calibration requirements for sets of sensors.

15. A method according to claim 1 wherein, as brain activity indexes, electromagnetic variables describing brain activity or variables derived from such electromagnetic variables are used.

16. A method according to claim 15 wherein the electromagnetic variables include potentials and/or currents and/or magnetic fields, measured extra- and/or intracranially.

17. A method according to claim 15, wherein brain activity indexes include frequencies of electromagnetic variables and/or coefficients of a representation of a time-series of electromagnetic variables in a Fourier- or wavelet- or Karhunen-Loeve-basis, and/or stochastic indexes.

18. A method according to claim 1, wherein target dynamics is calculated from the behavioral target by means of a behavioral model.

19. A method according to claim 1, wherein the influence of an exogenous electric and/or magnetic field on the brain activity of a specific user is determined.

20. A method according to claim 1, wherein a test signal is calculated, where the test signal is an exogenous electric and/or magnetic field applied to the user, whereby his/her brain activity is measured, and the brain activity measured during or after application of the test signal is evaluated together with previous brain activity, and by means of the result of this evaluation sets of parameters plus endogenous inputs plus influences of the test signal on the respective mode are determined, which are compatible with the data measured.

21. A method according to claim 20, wherein the test signal is modified and reapplied until the set of parameters, endogenous inputs and influence of the test signal on the considered mode is uniquely determined, whereby the mode is calibrated.

22. A method according to claim 20, wherein the calibration of brain activity is performed by calibration of modes and of interactions between modes, the former or both taking exogenous input into account.

23. A method according to claim 22, wherein the test signal has linear or nonlinear and/or chaotic and/or stochastic and/or non-stationary components.

24. A method according to claim 1, wherein a fast calibration selects a set of parameters, endogenous inputs, exogenous inputs compatible with the measured data, after the transmission of one test signal or a few test signals.

25. A method according to claim 24, wherein, using the calibrated brain activity model, it is calculated, which dynamics of a control variable is suitable to transform the factual dynamics of the brain activity indexes into the target dynamics, whereby the control variable represents the exogenous input within the brain activity model, which is related to exogenous magnetic and/or electric fields.

26. A method according to claim 25, wherein the to-be-calculated dynamics of a control variable is simplified by application of signals with known effects.

27. A method according to claim 26, wherein users are identified on the basis of such calibration data, which are stable over time.

28. A method according to claim 26, wherein calibration steps and modification steps are performed for groups of sensors and transmitters sequentially or in parallel.

29. A method according to claim 26, wherein bifurcation points are controlled, and thereby, amongst other effects, inactive modes are activated.

30. A method according to claim 1, wherein in addition to the magnetic and/or electric fields sensory inputs and/or biochemical active compounds are used.

31. A method according to claim 1, wherein, by virtue of a suitable repetition rate, brain activity is modified or stabilized beyond the duration of the application.

32. A method according to claim 1, wherein for the measurement of electromagnetic brain activity several sensors are interconnected.

33. A method according to claim 1, wherein a transmitter is used for generating the fields, the position and orientation of which can be altered.

34. A method according to claim 1, wherein for generating the fields several transmitters are used, several of which are interconnected.

35. A method according to claim 1, wherein the behavioral target includes one or more of the following behavioral targets:
alteration or sustenance of perception and/or perceptive capacity and/or perceptive capability,
alteration or sustenance of action and/or action capacity and/or action capability,
alteration or sustenance of reaction speed,
alteration or sustenance of activation and/or activation capacity,
alteration or sustenance of motivation and/or motivation capacity and/or motivation capability,
alteration or sustenance of attention and/or capacity for attention,
alteration or sustenance of memory and/or memory contents and/or memory retrieval
alteration or sustenance of learning and/or learning capacity and/or learning capability,
alteration or sustenance of consciousness,
alteration or sustenance of emotions and/or emotional capacity and/or emotional capability,
alteration or sustenance of appetencies and/or aversions,
alteration or sustenance of cognition and/or cognitive capacity and/or cognitive capability,
alteration or sustenance of behavioral sequences alteration or sustenance of behavioral correlations.

36. A method according to claim 35, wherein several not necessarily compatible behavioral targets are combined hierarchically.

37. A method according to claim 36, wherein the behavioral target of no-occurrence of seizures is given highest priority in this hierarchy.

38. A method according to claim 1, wherein several weighted behavioral models are used in parallel, whereby only those control- or regulation-steps are carried out, which are compatible with a majority of these models.

39. A method according to claim 1, wherein an external validation of behavioral modification/sustenance of behavior for the individual user is performed.

40. A method according to claim 1, wherein several weighted brain activity models are used in parallel, whereby only those control- or regulation-steps (S 3000) are carried out, which are compatible with a majority of these models.

41. A method according to claim 1, wherein generation of fields and measurements are carried out alternatingly.

42. A method according to claim 1, wherein generation of fields and measurements are performed simultaneously.

43. A method according to claim 1, wherein the method is carried out automatically.

44. An apparatus for achieving a given behavioral target, using a behavioral model which describes the correspondence between behavior and the dynamics of brain activity indexes, and using a brain activity model, which quantitatively describes brain activity, and from which brain activity indexes and the dynamics of said indexes can be derived, the apparatus comprising:
means for determining, using a behavioral model, which target dynamics of specific brain activity indexes corresponds to achieving the behavioral target,
means for generating exogenous electric and/or magnetic fields, and for applying these to a user, with at least one transmitter,
a measurement device for measuring electromagnetic brain activity with at least one sensor, and
means for calculating brain activity indexes, in order to achieve the target dynamics as part of an open or closed control loop.

45. Apparatus according to claim 44, characterized in that the measurement device has several sensors, the totality of which constitutes a sensor grid.

46. Apparatus according to claim 44, characterized in that the device for the generation of magnetic fields has several transmitters, the totality of which constitutes a transmitter grid.

47. Apparatus according to claim 44, characterized in that at least one computer is planned for, on which software modules for the implementation of the method according to one of the claims 1 to 43 are stored.

48. Apparatus according to claim 44, characterized in that electric and/or magnetic shielding is planned for every sensor and every transmitter.

49. Apparatus according to claim 44, characterized in that it is possible to mechanically decouple the measurement device from the other parts of the apparatus, such that the user can take the measurement device with him/her.

50. Apparatus according to claim 44, characterized in that the locations of sensors and transmitters are extracranial.

51. Apparatus according to claim 44, characterized in that sensors and transmitters are localized on the inside of a helmet which fits the shape of the cranium of the respective user.

52. Apparatus according to claim 44, characterized in that the transmitter grid and the sensor grid are superposed, such that there are transmitters in the vicinity of each sensor, and sensors in the vicinity of each transmitter.

53. Apparatus according to claim 44, characterized in that fittings in the transmitter grid are planned for, such that additional transmitters may be fixed therein, such that the transmitter density of a transmitter grid may be changed locally and/or the angle of individual transmitters with respect to the cranium of the user may be changed.

54. Apparatus according to claim 44, characterized in that the apparatus is decomposable into a head unit comprising sensors and transmitters, an intermediate unit and a basis unit, whereby the intermediate unit comprises a computer and software modules for performing the steps independent of calibration, and the basis unit comprises a computer and software modules for performing the steps dependent on calibration.
